(19) United States
(12) Patent Application Publication
(10) Pub. No.: US 2005/0244966 A1
(43) Pub. Date: Nov. 3, 2005

(54) Insulin producing cells

(75) Inventor: Shimon Efrat, Zikhron-Yaakov (IL)

Correspondence Address: Martin MOYNIHAN;c/o ANTHONY CASTORINA, SUITE 207, 2001 JEFFERSON DAVIS HIGHWAY, ARLINGTON, VA, 22202, US

(73) Assignee: Ramot At Tel Aviv University Ltd.

(21) Appl. No.: 11/174,480

(22) Filed: Jul. 6, 2005

Related U.S. Application Data
(63) Continuation-in-part of application No. PCT/GB04/00005, filed on Jan. 5, 2004.

(30) Foreign Application Priority Data
Jan. 6, 2003 (GB) 0300208.6

(51) Int. Cl. C12N 5/08

(52) U.S. Cl. 435/370

(57) ABSTRACT

The present invention relates to a method for modifying liver cells such that they produce and store insulin. In addition, the invention relates to these modified cells and their use in the treatment of insulin-dependant diabetes.

### RELATED PATENT APPLICATIONS

This application is a continuation-in-part of PCT Patent Application No. PCT/GB2004/000005, filed Jan. 5, 2004, which claims the benefit of U.K. Patent Application No. 0300208.6, filed Jan. 6, 2003, the contents of which are incorporated herein by reference in their entirety.

### FIELD OF THE INVENTION

The present invention relates to a method for modifying liver cells such that they produce and store insulin. In addition, the invention relates to these modified cells and their use in the treatment of insulin-dependant diabetes.

### BACKGROUND TO THE INVENTION

Immune-mediated (type 1) diabetes (or insulin dependant diabetes mellitus, IDDM) is an incurable disease of children and adults. The disease is characterised by an initial leukocyte infiltration into the pancreas that eventually leads to inflammatory lesions within islets, a process called “insulitis”. Overt disease is characterised by chronic autoimmune destruction of pancreatic beta cells in individuals. The progressive loss of pancreatic beta cells results in insufficient insulin production and thus impaired glucose metabolism with attendant complications.

Type 1 diabetes is currently managed by the administration of exogenous human recombinant insulin. Although insulin administration is effective in achieving euglycemia in most patients, it does not prevent the long term complications of the disease including ketosis and damage to small blood vessels which may affect eyesight, kidney function, blood pressure and can cause circulatory system complications.

Beta-cell replacement is considered the optimal treatment for type 1 diabetes. However, the availability of human organs for transplantation is limited. An effective cell replacement strategy depends on the development of an abundant supply of β cells and their protection from immune destruction. Reversible immortalization of differentiated β cells by conditional oncogene expression allowed the controlled expansion of murine cells in tissue culture, which were capable of replacing β cell function in vivo (1-3). However, this approach has not yet been successful with β cells from human islets, which tend to lose insulin expression during forced replication (4).

Study of pancreatic injury in animal models revealed that pancreatic duct epithelial cells are capable of islet neogenesis in adult animals. These cells may also be involved in pancreatic islet renewal in the absence of injury. Although epithelial cells isolated from pancreatic ducts were shown to differentiate into β. cells in culture (15,16), human progenitor cells capable of secreting insulin have not yet been isolated.

Although both U.S. patent application No. 20050053588 and 20050090465 teach converting liver stem and progenitor cells to insulin producing cells, no clinical data as to the quantities of insulin are incorporated. Furthermore, both U.S. patent application No. 20050053588 and 20050090465 do not teach of the combination of serum-free medium and activin to up-regulate insulin production following expression of pd-x.

U.S. patent application No. 20050053588 also does not provide any evidence that the liver progenitor cells are capable of producing a secretable form of insulin nor that its production is glucose regulated.

Accordingly, there is a need for an alternative source of insulin-producing cells for transplantation.

### SUMMARY OF THE INVENTION

The present invention demonstrates that human liver progenitor cells can be induced to differentiate into insulin-producing cells by modifying gene expression. The modified cells generated can produce, store, and release insulin in response to physiological glucose concentrations. They are able to restore and maintain euglycemia in a hyperglycemic immunodeficient mouse model.

Accordingly in a first aspect of the invention there is provided a human hepatic cell capable of endogenous insulin production wherein the insulin production comprises at least 50% of that of a normal human pancreatic islet.

As used herein, the phrase “endogenous insulin production” refers to an up-regulated expression (e.g., transcription, translation, processing) product of insulin from the hepatic cell genomic DNA of insulin. Using methods described herein the human hepatic cell is capable of producing quantities of insulin never before achieved such that the hepatic cell comprises at least 40%, at least 50% and preferably at least 60%, at least 70%, at least 80%, at least 90% or more say 100% of the amount of insulin that a normal human β cell does.

As used herein the phrase “normal β cell” refers to a cell that produces insulin in functional (i.e., insulin producing) isles of Langerhans in the pancreas.

The natural proliferative capacity of stem cells, both embryonic and from adult tissues provides a source of cells which can be induced to differentiate into insulin-producing cells. Recent studies showed that cultured mouse (5) and human (6) embryonic stem (ES) cells can differentiate at low frequencies into insulin-producing cells. By enrichment and expansion of nestin-positive cells, Lumelsky et al. significantly enhanced the generation of insulin-positive cells from mouse ES cell cultures (7).

Several groups have demonstrated the ability of stem/progenitor cells isolated from adult organs to differentiate along additional cell lineages (8, 9). These findings may reflect the presence of pluripotential cells in adult tissues, or the ability of committed stem/progenitor cells to transdifferentiate under suitable conditions.

Lineage switching in stem/progenitor cells may be stimulated by soluble factors, as well as master regulatory genes, which can activate hierarchically-determined cascades of gene expression in a dominant fashion.

Accordingly, a human “hepatic” cell refers to a cell which expresses at least one hepatic marker gene. Examples of hepatic marker genes include, but are not limited to, glycogen, dipeptidyl peptidase IV (DPPIV), gama-glutamyl transpeptidase (GGT), hepatocyte growth factor (HGF) and hepatocyte nuclear factors (HNFs). Such cells can be derived from embryonic stem cells or from progenitor cells derived from fetus or adult. Alternatively, cells may be fully differentiated along the hepatic lineage.

Preferably, the human hepatic cell is derived from a liver progenitor cell such as an epithelial progenitor cell. Such epithelial progenitor cells can be derived from suitably differentiated embryonic stem (ES) cell lines or from cells derived from a developing fetus.

Procedures for isolating and culturing unique populations of epithelial progenitor cells from human fetal liver (FH) have been described, for example, by Malhi et al. (17). These cells express markers of hepatocytes, bile duct cells and oval cells, and are capable of differentiating into mature hepatocytes in vivo (17).

Accordingly, in a preferred embodiment, the human hepatic cell is derived from epithelial progenitor cells from human fetal liver.

The use of progenitor cells derived from fetal human liver cells for developing a universal donor cell source for β-cell replacement offers several advantages, compared with efforts to obtain human insulin-producing cells from other sources, such as ES cells. Liver-derived cells express a number of transcription factors, the HNFs, which are needed in addition to Pdx1 for β-cell development and for maintaining the mature β-cell phenotype. In addition, HLA matching of banked fetal stem/progenitor liver cells, thereby increasing the likelihood of allograft survival, can be achieved far more readily than with the limited number of human ES cell lines.

Liver-derived insulin-producing cells may also have advantages over cells propagated from human islets. The absence of targets that activate autoimmune injury in β. cells may help avoid disease recurrence emanating from transplanted cell losses. Even if such antigens are expressed, liver-derived insulin-producing cells may be more resistant, compared with β. cells, to apoptosis induced by cytokines and free radicals, due to expression of higher levels of scavenging enzymes.

Modification of a human hepatic cell to induce insulin production can be through any method of modulating gene expression to activate the expression of insulin expression, or the expression of pro-insulin processing enzymes, either directly or through the modification of genes whose expression products are involved in activating expression of the insulin gene. Such genes include genes which induce expression of insulin mRNA, for example, genes encoding transcription factors such as PDX-1, BETA2, NKX6.1, neurogenin 3. Pro-insulin processing enzymes include, for example, prohormone convertase 1/3 and PC2. Other genes whose expression is associated with insulin expression include beta-cell protein islet amyloid polypeptide, chromogranin A, synaptogyrin 3.

A number of different methods of modulating gene expression will be recognised by those skilled in the art and include administering soluble factors which stimulate specific patterns of gene expression as well as activate expression of master regulatory genes, which can activate hierarchically-determined cascades of gene expression in a dominant fashion.

The parenchymal cells in liver share the same embryological origin as the pancreatic parenchymal cells i.e they are derived from the primitive foregut. In addition, mature hepatocytes and pancreatic β. cells manifest similarities in gene expression profiles, including genes encoding transcription factors, the glucose transporter GLUT2, and the glucose phosphorylating enzyme glucokinase (GK).

The HOX-like homeodomain transcription factor pancreatic duodenal homeobox 1 (Pdx1)(previously named ipf1) plays key roles in pancreas development (10), and is expressed in mature β cells as well, where it regulates expression of multiple genes, including insulin and the glucose transporter, GLUT2 (11). Forced Pdx1 expression in parenchymal mouse liver cells in vivo (12), and in rat enterocytes (13) and hepatic progenitor cells, designated “oval cells” (14), in vitro, was shown to activate □-cell genes, including insulin. Adult human liver cells have also been manipulated so that they are capable of insulin production (14b).

As demonstrated herein, Pdx1 expression activates numerous β-cell genes in human liver cells. Accordingly in one embodiment the human hepatic cell in accordance with the invention is modified to express Pdx 1. Suitably, the term “Pdx 1” refers to human Pdx 1 or homologues, orthologues, derivatives or variants thereof. As described herein, Pdx 1 can also refer to a rodent homologue.

In a particularly preferred embodiment, insulin production in the modified cell in accordance with the first aspect of the invention is glucose regulated. As used herein, insulin production may refer to insulin transcription, translation, processing, secretion or a combination thereof.

In another embodiment, the human hepatic cell can be further modified to enhance its capability to grow or survive and therefore be more likely to provide effective insulin production when introduced in vivo. Such further modifications can include modifications to immortalise a cell, or modifications to enhance immune tolerance etc.

Suitable methods for immortalization are known to those skilled in the art. One such method, which is exemplified herein, is immortalization following retroviral introduction of the catalytic subunit of the human telomerase gene (FH-hTERT) (18). As demonstrated herein, the replication potential of FH-hTERT cells was greatly enhanced, without evidence for neoplastic cell transformation, in either in vitro or in vivo assays.

Accordingly, in a particularly preferred embodiment, the human hepatic cell is a modified FH-hTERT cell.

In a second aspect of the invention there is provided a human hepatic cell capable of endogenous insulin production activatable by a combination of activin A and serum free medium.

According to the second aspects of the present invention the cells are activated to increase the amount of endogenous insulin by incubation in a serum-free medium (SFM) together with activin. Preferably, prior to the incubation, the cells of the present invention are forced to express PD-X by the introduction of the pd-x construct as described hereinabove.

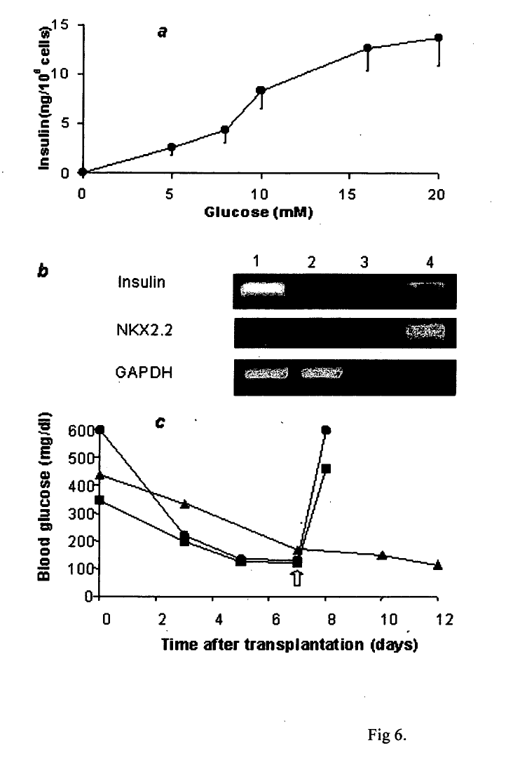

Serum-free medium may comprise culturing medium (e.g., DMEM), as well as other specific factors such as insulin (10 μg/ml), transferrin (5.5 μg/ml), and selenium (5 ng/ml; ITS, Sigma-Aldrich, Steinheim, Germany) but does not comprise whole serum (which without being bound by theory comprise agents which inhibit the insulin up-regulatory activity of Activin A). Serum-free medium was shown to enhance beta cell specific gene patterns by the up-regulation of insulin mRNA, PC2 mRNA and NKX2.2 mRNA (FIG. 6b).

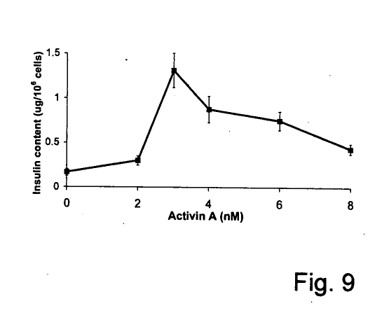

Addition of activin A (Cytolab/PreproTech Asia, Rehovot, Israel) in regular medium (i.e. comprising serum) at a concentration range between 1-8 nM was shown to further increase insulin content. A maximal increase in insulin content was obtained at 3 nM activin (FIG. 9).

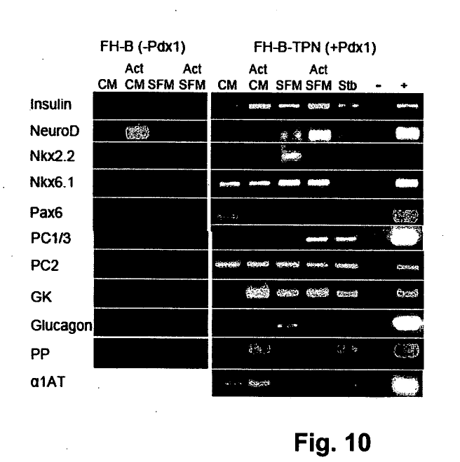

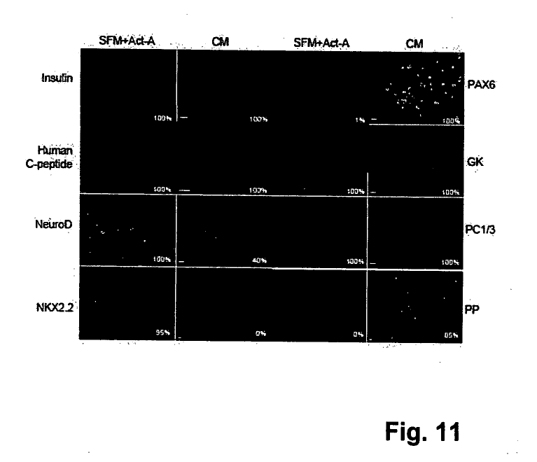

According to this aspect of the present invention, the cells are incubated with serum-free medium in the presence of activin A. Thus, a preferred culturing protocol of hepatic cells following introduction of the pd-x construct may comprise a 3-day Act-A treatment in SFM preceded by a 6-day incubation in SFM in the absence of Act-A (Table 4 of the Examples section below). Besides increasing insulin content in the hepatic cells, this treatment was shown to promote differentiation towards a beta cell by both up-regulation of beta-cell specific genes and the down-regulation of liver cell specific genes as shown in FIG. 10. Other soluble factors suitable for beta cell differentiation promotion may include betacellulin (BTC, R&D Systems, Minneapolis, Minn.), nicotinamide (NA, Sigma, Aldrich), exendin-4 (Sigma, Aldrich) and hepatocyte growth factor (HGF,Sigma, Aldrich).

In a third aspect of the invention, there is provided a method of up-regulating endogenous insulin production in a hepatic cell, the method comprising: (a) genetically modifying the hepatic cell to express at least one beta cell gene in the hepatic cell; and (b) culturing the genetically modified hepatic cell with serum-free medium and activin A.

As used herein the phrase “at least one beta cell gene” refers to any genes whose expression is normally detectable in pancreatic beta cells and is associated with insulin expression. In particular, beta cell genes include the transcription factors PD-X, BETA2, NKX6,1 and neurogenin 3, whose expression induce insulin mRNA expression, pro-insulin processing enzymes (prohormone convertase 1/3 and PC2), β-cell protein islet amyloid polypeptide, chromogranin A and/or synaptogyrin 3. In a preferred embodiment the beta cell gene is pd-x and the hepatic cell is an isolated hepatic progenitor cell. Suitably, the up-regulation of Pdx 1 is through the introduction of a nucleic acid construct (vector) for expressing Pdx 1 into the isolated hepatic progenitor cell followed by incubation of the cells in serum free medium and activin.

Preferably, following the introduction of the nucleic acid construct, hepatic cells which express at least one beta cell gene are selected and isolated. Typically the cells are then grown and expanded in serum-containing medium (e.g, fetal bovine serum), following which the cells are incubated in serum free medium and activin.

In a preferred embodiment, the construct for expressing Pdx 1 is a lentivirus. Suitably, the lentivirus is constructed using pONY4G SIN-MunI vector. The viral construct expressing Pdx 1 may be further modified, for example by pseudotyping. In the particular embodiment described herein, the pONY4-Pdx1 construct was pseudotyped by cotransfecting with the pONY3.1 plasmid (28) encoding viral gag/pol, and the pMD.G plasmid (30) encoding pseudotyped vesicular stomatitis virus envelope protein.

In another embodiment, the method in accordance with the third aspect of the invention further comprises immortalising said cells by introduction of expression of a telomerase gene.

In a fourth aspect there is provided a modified human cell in accordance with any embodiment of the first and second aspect of the invention for use as a medicament.

In a fifth aspect there is provided a method of treatment of type 1 diabetes comprising transplantation of at least one human hepatic cell as described herein, thereby treating type 1 diabetes.

### DETAILED DESCRIPTION OF THE INVENTION

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods. See, generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc.; as well as Guthrie et al., Guide to Yeast Genetics and Molecular Biology, Methods in Enzymology, Vol. 194, Academic Press, Inc., (1991), PCR Protocols: A Guide to Methods and Applications (Innis, et al. 1990. Academic Press, San Diego, Calif.), McPherson et al., PCR Volume 1, Oxford University Press, (1991), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.). These documents are incorporated herein by reference.

As used herein, “stem cells” are cells which retain their characteristic pluripotency or multipotency i.e. their ability to give rise to all cell types or more than one differentiated cell type.

By “progenitor cell” is meant a cell having the capacity to create progeny that are more differentiated than itself and which retains the capacity to replenish a pool of progenitors.

The terms “differentiated” or “differentiation status” when referring to a cell means cells that have begun to or have partially or completely developed into cells with a defined phenotype. The characteristic phenotypes of particular differentiated cell types are dependent on the particular cell type and are recognized to those skilled in the art. Accordingly, “hepatic” cells are cells that have partially or completely developed into cells with a hepatic phenotype characterized by the expression of hepatic marker genes.

The term “expression” refers to the transcription of a gene's DNA template to produce the corresponding mRNA and translation of this mRNA to produce the corresponding gene product (i.e., a peptide, polypeptide, or protein). The term “modified gene expression” or “altered gene expression” refers to inducing/increasing or inhibiting/blocking the transcription of a gene in response to a treatment where such induction/increase or inhibition/blocking is compared to the amount of gene expression in the absence of said treatment.

An “insulin producing” cell is a cell that generates insulin. This can be stored and secreted from cells and, in particular, can be secreted as a result of exposure to glucose.

Methods of determining insulin production in a cell are well known to those skilled in the art and include methods as described, for example, in Lumelsky et al (7) which include, RT-PCR, immunocytochemistry, immunostaining, immunoblotting etc. As described above, it is desirable that insulin production is glucose regulated. Methods for determining glucose regulation of insulin production are described herein and by Lumelsky et al. (7).

A “modified cell” in the context of the present invention is one that has been altered so as to be insulin producing. Suitably the cell is modified to have altered gene expression through the introduction of expression vectors comprising a nucleic acid encoding the gene of interest. In the context of the present invention, a gene of interest is one which modifies a hepatic cell to induce expression of insulin (endogenous) either directly or indirectly through a cascade of regulatory gene expression events. In a particular embodiment of the present invention, the gene of interest is the homeobox gene Pdx 1.

As used herein, a “vector” may be any agent capable of delivering or maintaining nucleic acid in a host cell, and includes viral vectors, plasmids, naked nucleic acids, nucleic acids complexed with polypeptide or other molecules and nucleic acids immobilised onto solid phase particles.

A “nucleic acid”, as referred to herein, may be DNA or RNA, naturally-occurring or synthetic, or any combination thereof. Nucleic acids encoding a gene of interest may be constructed in such a way that it may be translated by the machinery of the cells of a host organism. Thus, natural nucleic acids may be modified, for example to increase the stability thereof. DNA and/or RNA, but especially RNA, may be modified in order to improve nuclease resistance. For example, known modifications for ribonucleotides include 2′-O-methyl, 2′-fluoro, 2′-NH₂, and 2′-O-allyl. Modified nucleic acids may comprise chemical modifications which have been made in order to increase the in vivo stability of the nucleic acid, enhance or mediate the delivery thereof, or reduce the clearance rate from the body. Examples of such modifications include chemical substitutions at the ribose and/or phosphate and/or base positions of a given RNA sequence. See, for example, WO 92/03568; U.S. Pat. No. 5,118,672; Hobbs et al., (1973) Biochemistry 12:5138; Guschlbauer et al., (1977) Nucleic Acids Res. 4:1933; Schibaharu et al., (1987) Nucleic Acids Res. 15:4403; Pieken et al., (1991) Science 253:314, each of which is specifically incorporated herein by reference.

The terms “variant” or “derivative” in relation to a Pdx 1 polypeptide includes any substitution of, variation of, modification of, replacement of, deletion of or addition of one (or more) amino acids from or to the polypeptide sequence of any mammalian Pdx 1 sequence. Preferably, nucleic acids encoding Pdx 1 are understood to comprise variants or derivatives thereof.

Vectors for Gene Delivery or Expression

To generate cells expressing an exogenous gene, polypeptides such as Pdx 1 can be delivered by viral or non-viral techniques.

Non-viral delivery systems include but are not limited to DNA transfection methods. Here, transfection includes a process using a non-viral vector to deliver a gene to a target mammalian cell.

Typical transfection methods include electroporation, nucleic acid biolistics, lipid-mediated transfection, compacted nucleic acid-mediated transfection, liposomes, immunoliposomes, lipofectin, cationic agent-mediated, cationic facial amphiphiles (CFAs) (Nature Biotechnology 1996 14; 556), multivalent cations such as spermine, cationic lipids or polylysine, 1,2,-bis (oleoyloxy)-3-(trimethylammonio) propane (DOTAP)-cholesterol complexes (Wolff and Trubetskoy 1998 Nature Biotechnology 16: 421) and combinations thereof.

Viral delivery systems include but are not limited to adenovirus vectors, adeno-associated viral (AAV) vectors, herpes viral vectors, retroviral vectors, lentiviral vectors or baculoviral vectors, venezuelan equine encephalitis virus (VEE), poxviruses such as: canarypox virus (Taylor et al 1995 Vaccine 13:539-549), entomopox virus (Li Y et al 1998 XII^{th} International Poxvirus Symposium p 144. Abstract), penguine pox (Standard et al. J Gen Virol. 1998 79:1637-46) alphavirus, and alphavirus based DNA vectors.

A detailed list of retroviruses may be found in Coffin et al (“Retroviruses” 1997 Cold Spring Harbour Laboratory Press Eds: J M Coffin, S M Hughes, H E Varmus pp 758-763).

Lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

A distinction between the lentivirus family and other types of retroviruses is that lentiviruses have the capability to infect both dividing and non-dividing cells (Lewis et al 1992 EMBO. J 11: 3053-3058; Lewis and Emerman 1994 J. Virol. 68: 510-516). In contrast, other retroviruses—such as MLV—are unable to infect non-dividing cells such as those that make up, for example, muscle, brain, lung and liver tissue.

The vector encoding Pdx1 may be configured as a split-intron vector. A split intron vector is described in PCT patent applications WO 99/15683 and WO 99/15684.

If the features of adenoviruses are combined with the genetic stability of retroviruses/lentiviruses then essentially the adenovirus can be used to transduce target cells to become transient retroviral producer cells that could stably infect neighbouring cells. Such retroviral producer cells engineered to express Pdx1 can be implanted in organisms such as animals or humans for use in the treatment of diabetes.

Pox viruses are engineered for recombinant gene expression and for the use as recombinant live vaccines. This entails the use of recombinant techniques to introduce nucleic acids encoding foreign antigens into the genome of the pox virus. If the nucleic acid is integrated at a site in the viral DNA which is non-essential for the life cycle of the virus, it is possible for the newly produced recombinant pox virus to be infectious, that is to say to infect foreign cells and thus to express the integrated DNA sequence. The recombinant pox virus prepared in this way can be used as live vaccines for the prophylaxis and/or treatment of pathologic and infectious disease.

Expression of Pdx1 in recombinant pox viruses, such as vaccinia viruses, requires the ligation of vaccinia promoters to the nucleic acid encoding Pdx1. Plasmid vectors (also called insertion vectors), have been constructed to insert nucleic acids into vaccinia virus through homologous recombination between the viral sequences flanking the nucleic acid in a donor plasmid and homologous sequence present in the parental virus (Mackett et al 1982 PNAS 79: 7415-7419). One type of insertion vector is composed of: (a) a vaccinia virus promoter including the transcriptional initiation site; (b) several unique restriction endonuclease cloning sites located downstream from the transcriptional start site for insertion of nucleic acid; (c) nonessential vaccinia virus sequences (such as the Thymidine Kinase (TK) gene) flanking the promoter and cloning sites which direct insertion of the nucleic acid into the homologous nonessential region of the virus genome; and (d) a bacterial origin of replication and antibiotic resistance marker for replication and selection in E. Coli. Examples of such vectors are described by Mackett (Mackett et al 1984, J. Virol. 49: 857-864).

The isolated plasmid containing the nucleic acid to be inserted is transfected into a cell culture, e.g., chick embryo fibroblasts, along with the parental virus, e.g., poxvirus. Recombination between homologous pox DNA in the plasmid and the viral genome respectively results in a recombinant poxvirus modified by the presence of the promoter-gene construct in its genome, at a site which does not affect virus viability.

As noted above, the nucleic acid is inserted into a region (insertion region) in the virus which does not affect virus viability of the resultant recombinant virus. Such regions can be readily identified in a virus by, for example, randomly testing segments of virus DNA for regions that allow recombinant formation without seriously affecting virus viability of the recombinant. One region that can readily be used and is present in many viruses is the thymidine kinase (TK) gene. For example, the TK gene has been found in all pox virus genomes examined [leporipoxvirus: Upton, et al J. Virology 60:920 (1986) (shope fibroma virus); capripoxvirus: Gershon, et al J. Gen. Virol. 70:525 (1989) (Kenya sheep-1); orthopoxvirus: Weir, et al J. Virol 46:530 (1983) (vaccinia); Esposito, et al Virology 135:561 (1984) (monkeypox and variola virus); Hruby, et al PNAS, 80:3411 (1983) (vaccinia); Kilpatrick, et al Virology 143:399 (1985) (Yaba monkey tumour virus); avipoxvirus: Binns, et al J. Gen. Virol 69:1275 (1988) (fowlpox); Boyle, et al Virology 156:355 (1987) (fowlpox); Schnitzlein, et al J. Virological Method, 20:341 (1988) (fowlpox, quailpox); entomopox (Lytvyn, et al J. Gen. Virol 73:3235-3240 (1992)].

In vaccinia, in addition to the TK region, other insertion regions include, for example, HindIII M.

In fowlpox, in addition to the TK region, other insertion regions include, for example, BamHI J [Jenkins, et al AIDS Research and Human Retroviruses 7:991-998 (1991)] the EcoRI-HindIII fragment, BamHI fragment, EcoRV-HindIII fragment, BamHI fragment and the HindIII fragment set forth in EPO Application No. 0 308 220 A1. [Calvert, et al J. of Virol 67:3069-3076 (1993); Taylor, et al Vaccine 6:497-503 (1988); Spehner, et al (1990) and Boursnell, et al J. of Gen. Virol 71:621-628 (1990)].

In swinepox preferred insertion sites include the thymidine kinase gene region.

A promoter can readily be selected depending on the host and the target cell type. Preferably, the promoter is active in hepatic cells. For example the promoter may be a hepatic cell specific promoter such as that used in the Examples below (phosphoglycerate kinase 1 promoter). Alternatively, the promoter may be a constitutive promoter (i.e. capable of directing high level of gene expression in a plurality of tissues). For example in poxviruses, pox viral promoters should be used, such as the vaccinia 7.5K, or 40K or fowlpox C1. Artificial constructs containing appropriate pox sequences can also be used. Enhancer elements can also be used in combination to increase the level of expression. Furthermore, the use of inducible promoters, which are also well known in the art, are preferred in some embodiments.

Foreign gene expression can be detected by enzymatic or immunological assays (for example, immuno-precipitation, radioimmunoassay, or immunoblotting). Naturally occurring membrane glycoproteins produced from recombinant vaccinia infected cells are glycosylated and may be transported to the cell surface. High expressing levels can be obtained by using strong promoters.

Pseudotyping

In the design of retroviral vector systems it is desirable to engineer particles with different target cell specificities to the native virus, to enable the delivery of genetic material to an expanded or altered range of cell types. One manner in which to achieve this is by engineering the virus envelope protein to alter its specificity. Another approach is to introduce a heterologous envelope protein into the vector particle to replace or add to the native envelope protein of the virus.

The term pseudotyping means incorporating in at least a part of, or substituting a part of, or replacing all of, an env gene of a viral genome with a heterologous env gene, for example an env gene from another virus. Pseudotyping is not a new phenomenon and examples may be found in WO 99/61639, WO-A-98/05759, WO-A-98/05754, WO-A-97/17457, WO-A-96/09400, WO-A-91/00047 and Mebatsion et al 1997 Cell 90, 841-847.

Pseudotyping can improve retroviral vector stability and transduction efficiency. A pseudotype of murine leukemia virus packaged with lymphocytic choriomeningitis virus (LCMV) has been described (Miletic et al (1999) J. Virol. 73:6114-6116) and shown to be stable during ultracentrifugation and capable of infecting several cell lines from different species.

In the present invention the vector system may be pseudotyped.

Hepatic Cells

Preferably, the human hepatic cell is derived from a liver progenitor cell such as an epithelial progenitor cell. Such epithelial progenitor cells can be derived from suitably differentiated embryonic stem (ES) cell lines or from cells derived from a developing fetus.

Stem cells are undifferentiated, primitive cells with the ability both to multiply and differentiate into specific kinds of cells. Mammalian stem cells can be pluripotent cell lines derived from mammalian embryos, such as ES, EG or EC cells, or can be multipotent and derived from adults.

Embryonic stem (ES) cells are stem cells derived from the pluripotent inner cell mass (ICM) cells of the pre-implantation, blastocyst-stage embryo. Outgrowth cultures of blastocysts give rise to different types of colonies of cells, some of which have an undifferentiated phenotype. If these undifferentiated cells are sub-cultured onto feeder layers they can be expanded to form established ES cell lines that seem immortal. These pluripotent stem cells can differentiate in vitro into a wide variety of cell types representative the three primary germ layers in the embryo. Methods for deriving ES cells are known for example from Evans et al. 1981; Nature; 29; 154-156.

Embryonic germ (EG) cell lines are derived from primordial germ cells. Methods for the isolation and culture of these cells are described, for example, by McLaren et al. Reprod. Fertil. Dev 2001; 13 (7-8):661-4. Other types of stem cells include embryonal carcinoma cells (EC) (as reviewed, for example, in Donovan and Gearhar, Nature 2001; Insight review article p 92-97).

Other types of stem cells include cells having haploid genomes as described, for example, in WO 01/32015.

Methods for isolating human pluripotent stem cells are described, for example, by Trounson, A. O. Reprod. Fertil. Dev 2001; 13 (7-8): 523-32. Isolation requires feeder cells (and 20% fetal calf serum) or conditioned medium from feeder cells. Further methods for producing pluripotent cells are known from WO 01/30978 where the derivation of pluripotent cells from oocytes containing DNA of all male or female origin is described. In addition, stem cell-like lines may be produced by cross species nuclear transplantation as described, for example, in WO 01/19777, by cytoplasmic transfer to de-differentiate recipient cells as described, for example, in WO 01/00650 or by “reprogramming” cells for enhanced differentiation capacity using pluripotent stem cells (see WO 02/14469).

Methods of immortalizing cells to increase their proliferative potential in vitro and in vivo are described, for example, in Yeager et al., Current opinion in Biotechnology, 1999, 10: 465-469. As described herein, a preferred method involves forced expression of the enzyme telomerase.

As mentioned herein above, the hepatic cells of the present invention may be derived from a developing fetus. Procedures for isolating unique populations of epithelial progenitor cells from human fetal liver (FH) have been described, for example, by Malhi et al. (17). These cells express markers of hepatocytes, bile duct cells and oval cells, and are capable of differentiating into mature hepatocytes in vivo (17).

Stem Cell Culture

Cell culture conditions may be modified to favour maintenance of the cells in an undifferentiated state. If conditions are not carefully selected, stem cells may follow their natural capacity to differentiate into other cells. ES cells, for example, may differentiate into cells resembling those of extraembryonic lineages. Few of the factors that regulate self-renewal of pluripotent stem cells are currently known. Typically, pluripotent stem cell lines are isolated and maintained on mitotically inactive feeder layers of fibroblasts.

Typically, culture systems for ES cells comprise the use of media such as Dulbecco's modified Eagle's medium (DMEM) as a basal media with the addition of amino acids and beta mercaptoethanol, serum supplementation (normally Fetal Calf Serum (FCS)), and a embryonic mesenchymal feeder cell support layer. Basal media and serum supplements can be obtained from a number of commercial sources. However, any media or serum is subject to variability and even small variations can affect the ES cell culture conditions.

Cells maintained in their undifferentiated state may be subjected to control differentiating conditions to generate cells of the desired somatic lineage. Cultured stem cells can be induced to differentiate by separation of stem cells from feeder cells or by growth of stem cell colonies in suspension culture to form embryoid bodies which upon dissociation can be plated to yield differentiating cells. Conditions for obtaining differentiated cultures of somatic cells from ES cells are described, for example, in PCT/AU99/00990. Leukaemia inhibitory factor (LIF) has been identified as one of the factors that can maintain pluripotent stem cells; LIF can replace the requirement for feeder cells for murine ES cells (see Nichols et al.; (1990) Development 110; 1341-1348).

Methods of isolating and culturing fetal hepatic progenitor cells are described in the Examples section hereinbelow.

Differentiation of FH-B-TPN Cells Towards the Beta-Cell Phenotype

Differentiation of FH-B-TPN cells towards the beta-cell phenotype (i.e. insulin producing) may be verified using standard molecular biology techniques as described in the Examples section hereinbelow. For example, to verify the transcriptional up-regulation of beta-cell specific genes and the down-regulation of non beta-cell specific genes, Nothern analysis and RT-PCR may be performed. Additionally Western analysis and histological assays may be used to verify the up-regulation of beta-cell specific proteins and the down-regulation of liver-specific proteins. Preferably, differentiation of the cells of the present invention towards the beta-cell phenotype are also verified using animal experiments, such as transplanting them into a diabetic animal model (e.g. streptozotocin treated mice) and subsequent monitoring of blood glucose levels.

### METHODS OF TREATMENT

By “treating” is meant ameliorating or preventing the development of a disorder (in which up-regulating insulin levels is therapeutically beneficial) in a subject or individual showing any of the symptoms associated with that disorder or a subject or individual known to be at risk from developing that disorder.

For type 1 diabetes, a number of methods for diagnosing an individual suffering from the disorder are well known.

The term “therapy” includes curative effects, alleviation effects, and prophylactic effects. The therapy may be on humans or animals.

In particular, therapy is the treatment of the T cell mediated autoimmune disease, type 1 diabetes.

In a preferred embodiment of the present invention the patient in need thereof is treated with hepatic cells capable of producing endogenous insulin using an ex-vivo gene therapy approach described herein. The hepatic cells may be fully differentiated hepatic autologous cells removed from the patient or hepatic cells derived from another source as described herein. Successful ex-vivo gene therapy directed to autologous liver has been described—e.g. [Grossman M. et al., Nat Genet. April 1994; 6(4):335-41].

Cells and pharmaceutical comprising cells of the invention are typically administered to the patient by intramuscular, intraperitoneal or intravenous injection, or by direct injection into the lymph nodes of the patient, preferably by direct injection into the lymph nodes. Typically from 10⁴ to 10⁸ treated cells, preferably from 10⁵ to 10⁷ cells, more preferably about 10⁶ cells are administered to the patient.

The routes of administration and dosages described are intended only as a guide since a skilled practitioner will be able to determine readily the optimum route of administration and dosage for any particular patient depending on, for example, the age, weight and condition of the patient. Preferably the pharmaceutical compositions are in unit dosage form. The present invention includes both human and veterinary applications.

The invention is further described, for the purposes of illustration only, in the following examples in which reference is made to the following Figures and Tables:

### BRIEF DESCRIPTION OF THE DRAWINGS

### LEGENDS TO FIGURES

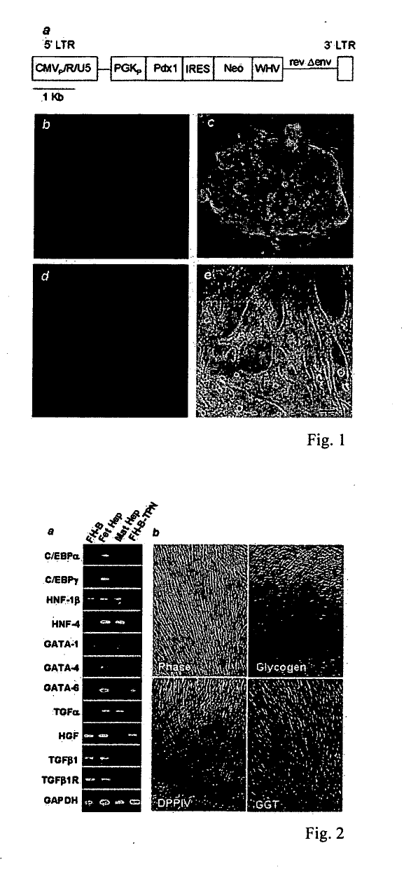

FIG. 1 Pdx1 induces insulin expression in FH-B-TPN cells.

a, diagram of the pONY4-Pdx1 viral vector. Transcription of the vector genome is driven by the human cytomegalovirus immediate-early enhancer/promoter (CMVP) fused to the R and U5 regions of the 5′ long terminal repeat (LTR) of EIAV. PGKP, phosphoglycerate kinase 1 promoter; IRES, internal ribosomal entry site; Neo, neomycin resistance gene; WHV post-transcriptional regulatory element of woodchuck hepatitis virus; rev, EIAV gene encoding the Rev protein; □env, mutated EIAV env gene.

b-e, histological analyses of FH-B-TPN cells. b, Nuclear PDX1 rhodamine-immunofluorescence; c, Phase contrast image of a ball-shaped cluster formed in a confluent culture; d, Cytoplasmic insulin Cy2-immunofluorescence; e, Phase contrast image of the same field shown in d. Panels b and d do not show the same field. FH-B cells were negative for staining for both proteins (data not shown). Bar represents 10 μm.

FIG. 2 Expression of liver genes in mature adult hepatocytes, primary fetal liver cells, FH-B cells and FH-B-TPN cells.

a, a panel of genes analyzed by RT-PCR. Primers for human GAPDH gene were used to monitor mRNA and cDNA quality.

b, histochemical studies of cultured FH-B-TPN cells showing flat epithelial cell morphology (phase contrast), and expression of glycogen (hepatocyte marker), DPPIV (bile canalicular marker) and GGT (biliary marker). Magnification is X90.

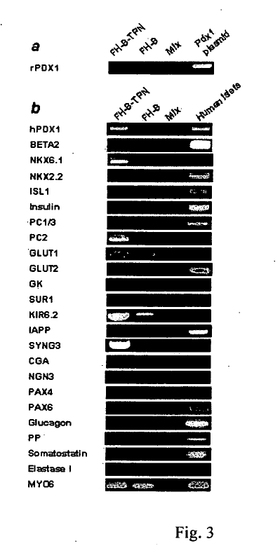

FIG. 3 Expression of transcripts of pancreatic genes in FH-B-TPN cells. mRNA extracted from FH-B-TPN and FH-B cells was subjected to RT-PCR analysis with primers for the indicated genes.

a, Analysis of expression of rat Pdx1, with plasmid DNA as positive control.

b, Analysis of expression of human pancreatic genes, with human islets as positive control. Primers for myosin 6 (MYO6) were used to monitor mRNA and cDNA quality. The amount of human islet cDNA used in the analysis with insulin primers was 1/10 of the amount used for the other genes. Mix, PCR reaction without cDNA.

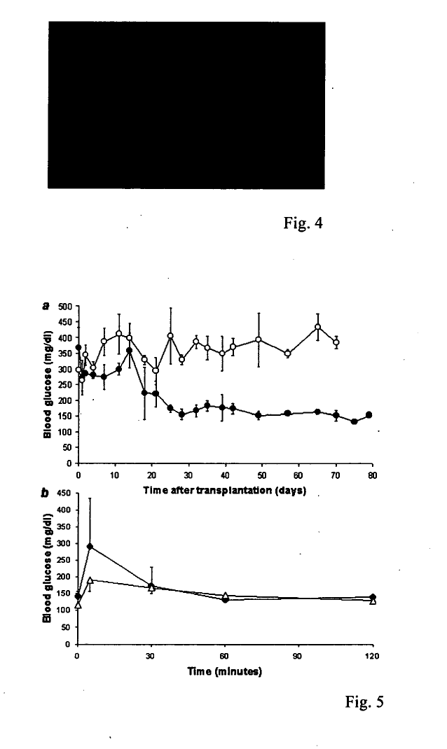

FIG. 4 Glucose-induced insulin secretion from FH-B-TPN cells during a 2-h static incubation. Values are mean±SEM of 9 replicate wells from 3 independent experiments.

FIG. 5 FH-B-TPN cells can replace β-cell function in NOD-scid STZ-induced diabetic mice.

a, Fed blood glucose levels. Closed circles, transplanted mice (n=3); open circles, untransplanted mice (n=4). Values are mean±SEM. The differences between the 2 groups were significant on days 25-70 post transplantation (P<0.0001).

b, GTT performed on the mice in a 70 days following transplantation. Circles, transplanted diabetic mice; triangles, non-diabetic NOD-scid mice. Values are mean±SEM. The differences between transplanted and non-diabetic mice were not significant at the 30-120 minute time points (P>0.2). Untransplanted diabetic mice were hyperglycemic (>350 mg/dl) at all time points (data not shown).

FIG. 6 Characterization of FH-B-TPN cells following a 6-day incubation in serum-free medium.

a, Glucose-induced insulin secretion during a 30-minute incubation. Values are mean±SEM of 3 replicate wells.

b, Expression of transcripts of pancreatic genes analyzed by RT-PCR. 1, serum-free medium; 2, regular medium; 3, mix; 4, human islets.

c, Cell transplantation under the renal capsule of NOD-scid STZ-induced diabetic mice. Each line represents fed blood glucose levels of a single mouse. The arrow marks left-kidney nephrectomy of the mice labeled by circles and squares, leading to hyperglycemia.

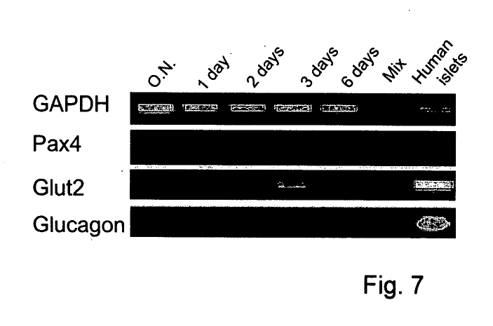

FIG. 7 Expression of transcripts of β-cell genes in FH-B-TPN cells treated with 4 nM activin A for the indicated time. mRNA extracted from the cells at the end of incubation was subjected to RT-PCR analysis with primers for the indicated genes. Human islet mRNA served as positive control. Primers for GAPDH were used to monitor mRNA and cDNA quality. Mix, PCR reaction without cDNA.

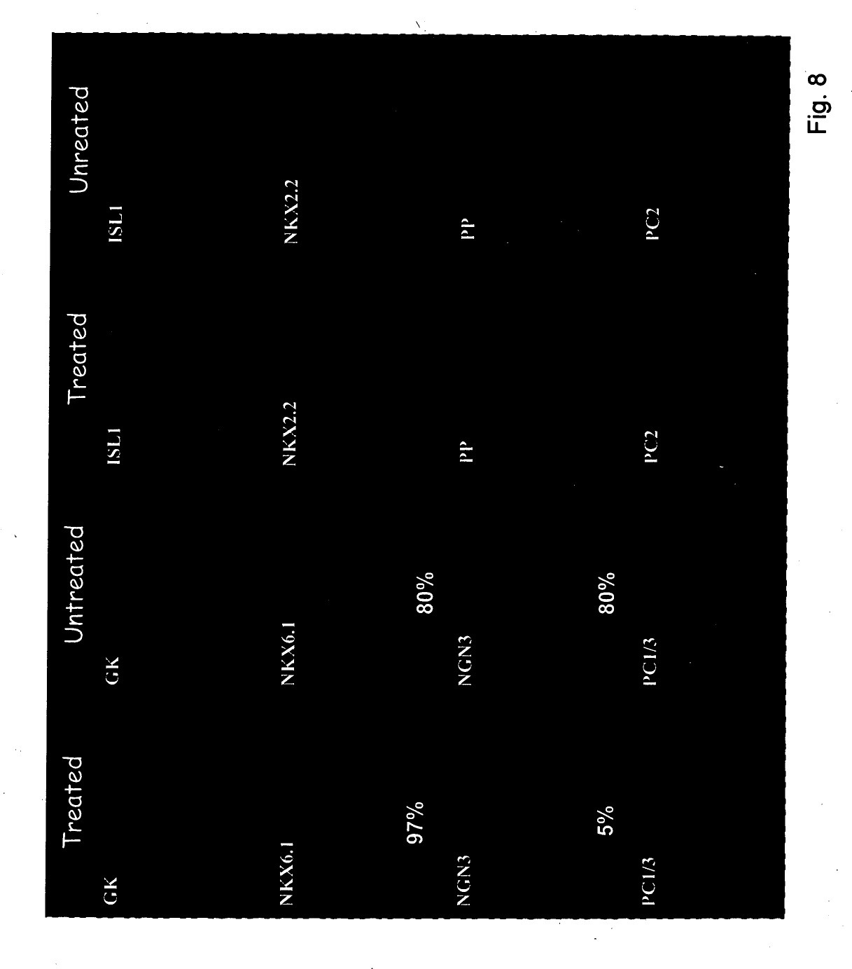

FIG. 8 Immunofluorescence analyses of FH-B-TPN cells incubated in regular medium (untreated) or following a 6-day incubation in serum-free medium supplemented with ITS and activin A (treated).

FIG. 9 A line graph illustrating the effect of Activin-A concentration on insulin content in FH-B-TPN cells. FH-B-TPN cells were incubated with the indicated concentrations of Act-A in CM for 6 days. Insulin levels in cell extracts were quantitated by RIA. Values are mean±SD (n=3).

FIG. 10 A photograph illustrating RT-PCR analysis of gene expression in FH-B and FH-B-TPN cells treated with various culture media. Cells were grown >7 days in CM, 3 days in CM containing Act-A, 6 days in SFM, 6 days in SFM followed by 3 days of Act-A in SFM, or tested for phenotypic stability (Stb) 10 days after shift from the last medium into CM. RNA extracted from the cells was analyzed by RT-PCR with the indicated primers, in comparison with a negative control (−,minus-template) and positive control (+, genomic DNA for alpha 1-antitrypsin and human islet RNA for the rest).

FIG. 11 Photographs illustrating immunofluorescence analyses of protein expression in FH-B-TPN cells treated with SFM (6days) followed by Act-A in SFM (3days) (Treated), compared with cells grown in CM (Untreated). Indicated antigens were visualized with Cy2-(green) and Cy3-(red) conjugated second antibodies. All nuclei were labeled blue with DAPI. The percent of positive cells shown on each panel is based on counting >300 cells in multiple fields. Bar=10 μm.

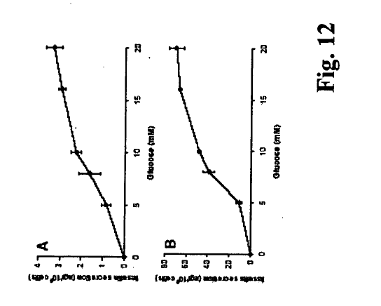

FIG. 12 Plot graphs illustrating glucose-induced insulin secretion in FH-B-TPN cells treated with Act-A.

a, Cells treated with Act-A in CM for 3 days

b, Cells treated with SFM for 6 days followed by a 3-day treatment with Act-A in SFM.

Insulin secretion was studied in KRB containing 0.5 mM IBMX and the indicated concentrations of glucose during a 30-min incubation. Insulin in the medium was quantitated by ELISA and normalized to cell number. Values are mean±SD (n=3).

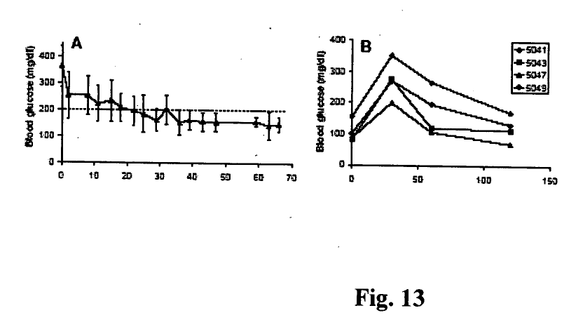

FIG. 13 Restoration of euglycemia in NOD-SCID mice transplanted with FH-B-TPN cells following treatment with Act-A in SFM. Mice, made diabetic by STZ treatment, were injected with 2×10⁶ cells at passage 17 under the left renal capsule. Fed blood glucose was measured twice a week.

a, Blood glucose levels. Values are mean±SD (n=7). The dashed line marks the upper end of normal fed blood glucose levels. Mice transplanted with 5×10⁶ FH-B cells died within 6 days following transplantation.

b, GTT performed on 4 of the 7 mice shown in FIG. 13a on day 65 post-transplantation. Each curve represents an individual mouse, identified by number. Two normal mice are included as controls.

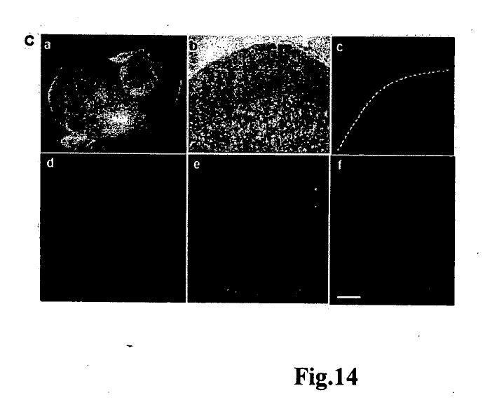

FIG. 14 Histological analyses of the transplanted cells.

a, kidney with transplanted cells seen at its top right corner;

b, hematoxylin and eosin staining of a kidney section showing the transplanted cells in the top half;

c, immunofluorescence analysis of adjacent section with BrdU antibody visualized with Cy3-conjugated second antibody. Nuclei are labeled blue with DAPI. The dashed line marks the boundary between the transplanted cells and the kidney parenchyma. A single nucleus labeled by BrdU is seen in the latter. Bar=50μm.

d, immunofluorescence analysis of adjacent section with insulin antibody visualized with Cy3-conjugated second antibody. Nuclei are labeled blue with DAPI.

e, immunofluorescence analysis of adjacent section with human C-Peptide antibody visualized with Cy3-conjugated second antibody. Nuclei are labeled blue with DAPI.

f, immunofluorescence analysis of adjacent section with HSP-27 antibody visualized with Cy3-conjugated second antibody. Nuclei are labeled blue with DAPI.

### EXAMPLES

### METHODS

Cell Culture.

FH cells were isolated and cultured as described (19), in Dulbecco's modified Eagle's medium containing 25 mM glucose and supplemented with 10% fetal bovine serum (FBS), 100 U/ml penicillin, 100 μg/ml streptomycin, 5 μM hydrocortisone, and 5 μg/ml insulin. A subclone of FH cells that was stably transduced with hTERT, designated FH-B-hTERT (FH-B), was cultured in the same medium, as described (20). FH-B cells transduced with the Pdx1 gene (see below), designated FH-B-TPN cells, were maintained in the same culture medium, with the exception of hydrocortisone and insulin (complete medium CM). For incubation in serum-free medium, the cells were placed in DMEM containing antibiotics, in the presence of 100 ng/ml insulin, 55 ng/ml transferrin, and 50 pg/ml selenium (ITS, Sigma). For all experiments activin A (Cytolab/PreproTech Asia, Rehovot, Israel) was added at a final concentration of 4 nM. except for those aimed at promoting differentiation of FH-B-TPN cells towards the beta-cell phenotype. There, addition of Act-A, as well as betacellulin (BTC, R&D Systems, Minneapolis, Minn.), nicotinamide (NA), exendin-4, and hepatocyte growth factor (HGF) (the last 3 compounds from Sigma-Aldrich) was at concentrations detailed herein below.

Construction of Pdx1 Lentivirus.

A Pdx1 lentivirus was generated using an equine infectious anemia virus (EIAV)-based vector (21,22). A fragment including rat Pdx1 cDNA (23) under control of the mouse phosphoglycerate kinase 1 promoter (PGKP; ref. 24), placed upstream of an encephalomyocarditis virus IRES (25), a neomycin resistance gene, and the post-transcriptional regulatory element of woodchuck hepatitis virus (WHV), was inserted between the XbaI and KpnI sites of the pONY4G SIN-MunI vector (Oxford Biomedica, Oxford, UK) (26). The long terminal repeats (LTR) of this vector are mutated by removal of the U3 element to prevent LTR-mediated transactivation of gene expression and to limit recombination into replication-competent virus. The WHV element increases nuclear RNA stability and the efficiency of its transport out of the nucleus (27). The pONY4-Pdx1 construct (FIG. 1a) was cotransfected with the pONY3.1 plasmid (26) encoding viral gag/pol, and the pMD.G plasmid (28) encoding pseudotyped vesicular stomatitis virus envelope protein, into 293T cells as described (28). The culture medium was collected 36 hours later and titrated in COS7 cells by counting geneticin (G418)-resistant colonies. FH-B cells were incubated overnight with Pdx1 lentivirus under 5:1 multiplicity of infection at 37° C. in medium containing 5 mM glucose. After 2 days the medium was switched to 25 mM glucose and FHB-TPN cells were selected with 200 □g/ml G418 for 16 days.

RT-PCR Analyses.

Total RNA was extracted from cultured cells, mature human hepatocytes (obtained from Incara Cell Technologies, North Carolina), and human pancreatic islets (obtained through the Juvenile Diabetes Research Foundation Islet Distribution Program), using commercial kits (High Pure RNA isolation kit, Roche Molecular Biochemicals, Mannhein, Germany; Trizol Reagent, Invitrogen Life Technologies, Carlsbad, Calif.).

Specific transcripts were analyzed with Promega (Madison, Wis.) RT-PCR kit or GeneAmp Gold RNA PCR kit (Perkin Elmer Corp., Indianapolis, Ind.) or Superscript III RT-PCR (Invitrogene Life Technologies, Carlsbad, Calif.) according to the manufacturers. The absence of DNA contamination in RNA samples was confirmed with PCR primers flanking an intron. cDNA was amplified for 40 cycles (94° C. for 45 sec; annealing under conditions indicated in Table 1 below for 45 sec; 72° C. for 40 sec), using the primer pairs listed in Table 1 below.

TABLE 1

| | Sense Primer | Antisense Primer | Annealing |
|---|---|---|---|
| Gene | (SEQ ID NO) | (SEQ ID NO) | temperature |
| Rat Pdx1 | CAAGGACCCATGCGCGTTCCAGC | GAACTCCTTCTCCAGCTCTAGCAGCTG | 60° C. |
| | | (2) | |
| Human | CAAGGACCCATGCGCGTTCCAGCGA | GAACTCCCTTCTCCAGCTCTAGCAGCTG | 60° C. |
| Pdx1 | (3) | (4) | |
| BETA 2 | CCTGAGCAGAACCAGGACATGCC | ATCAAAGGAAGGGCTGGTGCAATCA | 58° C. |
| | (5) | (6) | |
| NKX6.1 | CTCCTCCTCGTCCTCGTCGTCGTC | CTTGACCTGACTCTCTGTCATC | 60° C. |
| | (7) | (8) | |
| NKX2.2 | CGGACAATGACAAGGAGACCCCG | CGCTCACCAAGTCCACTGCTGCTGG | 65° C. |
| | (9) | (10) | |
| ISL1 | GTGCGGAGTGTAATCAGTATTTGG | GTCATCTCTACCAGTTGCTCCTTC | 58° C. |
| | (11) | (12) | |
| Insulin | GCTGCATCAGAAGAGGCCATCAGGC | GCGTCTAGTTGCAGTAGTTCTCCAG | 58° C. |
| | (13) | (14) | |
| PC1/3 | TTGGCTGAAAGAGAACGGGATACATCT | ACTTCTTTGGTGATTGCTTTGGCGGTG | 65° C. |
| | (15) | (16) | |
| PC2 | GCATCAAGCACAGACCTACACTCG | GAGACACAACCACCCTTCATCGTTC | 60° C. |
| | (17) | (18) | |
| Glut 1 | CTCACTGCTCAAGAAGACATGG | CTGGGTAACAGGGATCAAACAG | 65° C. |
| | (19) | (20) | |
| Glut 2 | GCCATCCTTCAGTCTCTGCTACTC | GCTATCATGCTCACATAACTCATCCA | 65° C. |
| | (21) | (22) | |
| GK | GACGAGTTCCTGCTGGAGTATGAC | GACTCGATGAAGGTGATCTCGCAGCTG | 65° C. |
| | (23) | (24) | |
| SUR1 | GTGCACATCCACCACAGCACATGGCTTC | GTGTCTTGAAGAAGATGTATCTCCTCAC | 62° C. |
| | (25) | (26) | |
| KIR6.2 | CGCTGGTGGACCTCAAGTGGC | CCTCGGGGCTGGTGGTCTTGCG | 65° C. |
| | (27) | (28) | |
| IAPP | GAGAGAGCCACTGAATTACTTGCC | CCTGACCTTATCGTGATCTGCCTGC | 65° C. |
| | (29) | (30) | |
| SYNG3 | GGTAGTGACTGTCTCGTTTCTGTC | AGCTATGCAGAGGGACTCCAACCTG | 60° C. |
| | (31) | (32) | |
| CGA | CGGACAGTTCCATGAAGCTCTC | GAGTCAGGAGTAGGAGACAAGG | 58° C. |
| | (33) | (34) | |
| NGN3 | ACTGAGCAAGCAGCGACGGAGTC | GCACCCACAGCCGAGCGACAGAC | 65° C. |
| | (35) | (36) | |
| PAX4 | CACCTCTCTGCCTGAGGACACGGTGAG | CTGCCTCATTCCAAGCCATACAGTAGTG | 60° C. |
| | (37) | (38) | |
| PAX6 | CAGTCACAGCGGAGTGAATCAGC | GCCATCTTGCGTAGGTTGCCCTG | 58° C. |
| | (39) | (40) | |
| Glucagon | GAATTCATTGCTTGGCTGGTGAAAGGC | CATTTCAAACATCCCACGTGGCATGCA | 60° C. |
| | (41) | (42) | |
| PP | CTGCTGCTCCTGTCCACCTGCGTG | CTCCGAGAAGGCCAGCGTGTCCTC | 60° C. |
| | (43) | (44) | |
| Somatostatin | CGTCAGTTTCTGCAGAAGTCCCTGGCT | CCATAGCCGGGTTTGAGTTAGCAGATC | 60° C. |
| | (45) | (46) | |
| Elastase 1 | GTGATGACAGCTGCTCACTGCGTG | CATCTCCACCAGCACACACCATGGTG | 60° C. |
| | (47) | (48) | |
| MYO6 | CTTGAGATGGAAGCAAAGAG | CTTCACTCTGGGCAATCCTCA | 58° C. |
| | (49) | (50) | |
| C/EBP/α | CAAGAAGTCGGTGGACAAGAAC | CCTCATCTTAGACGCACCAAGT | 58° C. |
| | (51) | (52) | |
| C/EBP/γ | GCAACGCCGAGAGAGGA | TGTCCTGCATTGTCGCC | 58° C. |
| | (53) | (54) | |
| HNF1β | GAAACAATGAGATCACTTCCTCC | CTTTGTGCAATTGCCATGACTCC | 56° C. |
| | (55) | (56) | |
| HNF4 | CTGCTCGGAGCCACAAAGAGATCCATG | ATCATCTGCCACGTGATGCTCTGCA | 58° C. |
| | (57) | (58) | |
| GATA-1 | CAGTCTTTCAGGTGTACCC | GAGTGATGATGAAGGCAGTGCAG | 56° C. |
| | (59) | (60) | |
| GATA-4 | TCCCTCTTCCCTCCTCAAAT | TTCCCCTAACCAGATTGTCG | 58° C. |
| | (61) | (62) | |
| GATA-6 | GAGTGGAAGGGAAGGGCGAG | GAAGAAGCACATGATTTGGGAC | 58° C. |
| | (63) | (64) | |
| TGFα | ATGGTCCCCTCGGCTGGA | GGCCTGCTTCTTCTGGCTGGCA | 58 |
| | (65) | (66) | |
| HGF | AGGAGCCAGCCTGAATGATGA | CCCTCTGATGTCCCAAGATTAGC | 56° C. |
| | (67) | (68) | |
| TGFβ1 | GCCCTGGACACCAACTGTTGCT | AGGCTCCAAATGTAGGGGCAGG | 58° C. |
| | (69) | (70) | |
| TGFβ1R | CGTGCTGACATCTATGCAAT | AGCTGCTCCATTGGCATAC | 54° C. |
| | (71) | (72) | |
| GAPDH | CCATGGAGAAGGCTGGGG | CAAAGTTGTCATGGATGACC | 58° C. |
| | (73) | (74) | |
| NKX6.1* | ACACGAGACCCACTTTTTCCG | TGCTGGACTTGTGCTTCTTCAAC | 59° C. |
| | (75) | (76) | |
| Neuro D* | AAGAACTACATCTGGGCTCTGTCG | GCTGAGGGGTCCATCAAAGG | 59.8° C. |
| | (77) | (78) | |
| α1AT* | GGCATCACTAAGGTCTTCAGCAATG | GAGCGAGAGGCAGTTATTTTTGG | 57.2° C. |
| | (79) | (80) | |
| NKX2.2* | TCTGAACCTTGGGAGAGGGC | GGTCATTTTGGCAACAATCACC | 54.7° C. |
| | (81) | (82) | |
| Insulin* | AACCAACACCTGTGCGGCTC | GGGCTTTATTCCATCTCTCTCGG | 61.1° C. |
| | (83) | (84) | |
| PC1/3* | CTCCTAAAAGACTTGCGGAATCAC | TCCACACAGGCACTAAGAAAGACTG | 52.1° C. |
| | (85) | (86) | |
| PC2* | GCGGGATTACCAGTCCAAGTTG | TGTGCTTTCAGAGATGTGGCG | 55.7° C. |
| | (87) | (88) | |
| GK* | TCACTGTGGGCGTGGATGG | ACCGAAAAACTGAGGGAAGAGG | 61.6° C. |
| | (89) | (90) | |
| PAX6* | GCCAAATGGAGAAGAGAAGAAAAAC | GTTGAAGTGGTGCCCGAGG | 57.8° C. |
| | (91) | (92) | |
| Glucagon* | CGTTCCCTTCAAGACACAGAGGAG | TCCCTGGCGGCAAGATTATC | 56.8° C. |
| | (93) | (94) | |
| PP* | CAATGCCACACCAGAGCAGATG | TGGGAGCAGGGAGCAAGC | 59° C. |
| | (95) | (96) | |

*Primers used in experiments aimed at promoting differentiation of FH-B-TPN cells towards the beta-cell phenotype

PCR products were separated by electrophoresis in 1.5%-2.5% agarose gels and visualized by ethidium bromide staining.

Insulin Secretion and Content.

Insulin secretion from FH-B-TPN cells was measured by static incubation as previously described (2). Cells were plated in 24-well plates at 10⁵ cells per well. The cells were preincubated for 1 hour in Krebs-Ringer buffer (KRB), followed by incubation for the indicated period of time in KRB containing 0.5 mM 1-isobutyl 3-methylxanthine (IBMX) and glucose at various concentrations. The cells were then extracted in acetic acid, and the amount of insulin in the buffer and cell extract was determined by radioimmunoassay (RIA) using the INSIK-5 kit (DiaSorin, Vercelli, Italy) according to the manufacturer. This assay has <20% crossreactivity with proinsulin. In addition, insulin content was determined using an ELISA kit (Diagnostic Systems Laboratories, Webster, Tex. or (Mercodia, Uppsala, Sweden), which recognizes only mature insulin. Insulin content was normalized to total cellular protein, measured by the Bio-Rad (Hercules, Calif.) Protein Assay kit. Human C-peptide in the cell extract was determined using a RIA kit (DiaSorin, Vercelli, Italy) or ELISA kit (Mercodia, Uppsala, Sweden) according to the manufacturers.

Cell Proliferation Assay.

[³H]thymidine incorporation was measured in 10⁴ cells during a 16-h pulse, as previously described (1).

Cell Transplantation.

Six-week-old nonobese diabetic severe combined immunodeficient (NOD-scid) female mice (Harlan, Jerusalem, Israel) were made hyperglycemic by i.p. injection of streptozotocin (STZ) at 180 μg per gr body weight. When blood glucose reached levels >300 mg/dl, mice were transplanted on the same day with 10⁷ FH-B-TPN cells in 0.5 ml PBS i.p. Blood glucose levels were monitored twice a week in samples obtained from the tail vein of fed mice using Accutrend strips (Roche). Serum insulin and human C-peptide levels were determined by RIA in blood samples obtained from the orbital plexus of fed mice, using the INSIK-5 and Double Antibody C-Peptide (EURO/DPC, Llanberis, UK) kits, respectively, according to the manufacturers. The human C-peptide kit had 0% cross reactivity with mouse C-peptide. For transplantation under the renal capsule, 2-3×10⁶ cells pre-incubated for 6 days in serum-free medium were placed in 50 μl PBS and injected in the left kidney using a 30-gauge needle. At the indicated time point the mice were anesthetized and subjected to left kidney nephrectomy. For some experiments, mice were injected with 100 μg 5-bromo-2-deoxyuridine (BrdU, Sigma-Aldrich) per gr body weight 6 hours prior to the nephrectomy. Mice were monitored one day later for changes in blood glucose levels.

Glucose Tolerance Test (GTT).

Mice fasted for 6 hours were injected i.p. with glucose in saline at 1 mg per gr body weight. Blood glucose levels were monitored at the indicated time points in samples obtained from the tail vein.

Histological Assays for Islet Gene Expression.

FH-B-TPN cells plated in 6-well plates on sterilized coverslips were fixed in 4% paraformaldehyde. For cytoplasmic antigens, cells were blocked for 10 min at room temperature in 5% bovine serum albumin, 5% FBS and 0.1% Triton X-100, and stained with the antibodies as detailed in Table 2 diluted in blocking solution, for 1 hour at room temperature.

TABLE 2

| | Cellular | Primary antibody | | | Secondary antibody | | |
|---|---|---|---|---|---|---|---|
| Antigen | location | Species | Dilution | Source | Label | Species | Dilution |
| *Insulin | cytoplasmic | mouse | 1:1000 | Sigma, St. Louis, MI | Cy3 | Goat | 1:200 |
| Insulin | cytoplasmic | G. pig | 1:1000 | Linco Res, St. Charles, MO | Cy2 | donkey | 1:400 |
| Pdx | nuclear | rabbit | 1:5000 | | rhodamine | donkey | 1:600 |
| *Human | cytoplasmic | mouse | 1:200 | Biodesign Int., Saco, ME | Cy3 | Goat | 1:200 |
| C- | | | | | | | |
| peptide | | | | | | | |
| ISL-1 | nuclear | mouse | 1:10 | DSHB, Iowa City, IA | rhodamine | donkey | 1:200 |
| *NeuroD | nuclear | goat | 1:250 | Santa Cruz Biotechnology, | Cy2 | Rabbit | 1:200 |
| | | | | Santa Cruz, CA | | | |
| *NKX2.2 | nuclear | mouse | 1:10 | DSHB, Iowa City, IA | Cy3 | Goat | 1:200 |
| NKX2.2 | nuclear | mouse | 1:10 | DSHB, Iowa City, IA | rhodamine | Goat | 1:200 |
| NKX6.1 | nuclear | rabbit | 1:200 | | rhodamine | donkey | 1:600 |
| NGN3 | nuclear | rabbit | 1:350 | | rhodamine | donkey | 1:600 |
| *PAX6 | nuclear | rabbit | 1:1000 | Chemicon, Temecula, CA | Cy2 | Rabbit | 1:200 |
| *GK | cytoplasmic | rabbit | 1:200 | M. Magnuson | Cy3 | Donkey | 1:500 |
| *PC1/3 | cytoplasmic | rabbit | 1:200 | D. Steiner | Cy3 | Donkey | 1:500 |
| PC1/3 | cytoplasmic | rabbit | 1:200 | D. Steiner | rhodamine | Donkey | 1:600 |
| *PP | cytoplasmic | rabbit | 1:500 | DAKO, Carpinteria, CA | Cy3 | Donkey | 1:500 |
| PP | cytoplasmic | rabbit | 1:500 | DAKO, Carpinteria, CA | rhodamine | Donkey | 1:600 |
| PC2 | cytoplasmic | rabbit | 1:1000 | | rhodamine | Donkey | 1:600 |
| *HSP-27 | nuclear | mouse | 1:50 | NeoMarkers, Fremont, CA | Cy3 | Goat | 1:200 |
| *BrdU | nuclear | mouse | 1:50 | BD Biosciences, San Jose CA | Cy3 | Goat | 1:200 |

*Used in experiments aimed at promoting differentiation of FH-B-TPN cells towards the beta-cell phenotype

For immunostaining of nuclear antigens, cells were permeabilized in 0.25% NP40 for 10 min at room temperature prior to blocking, followed by incubation with the primary antibody as detailed in Table 2 hereinabove. The stained cells were photographed under a Zeiss confocal microscope. FH-B, Cos7 or 293T cells were used as negative controls. Nuclei were visualized with DAPI (Roche) staining for 5 minutes at room temperature. Kidney tissue was fixed in 4% paraformaldehyde, embedded in paraffin, and sectioned. Sections were rehydrated, washed in PBS, and unmasked (if needed) in Unmasking Solution (Vector Laboratories, Burlingame, Calif.) according to the manufacturer. Sections were blocked for 2 h in 0.2% Tween 20 and 0.2% gelatin, incubated overnight at 4° C. with primary antibodies and 2 h at room temperature with the secondary antibodies as detailed in Table 2 hereinabove, stained with DAPI, and mounted. BrdU staining was performed as previously described (28b).

Histological Studies of Liver Gene Expression.

Cells were stained for glycogen, dipeptidyl peptidase IV (DPPIV), and γ-glutamyl transpeptidase (GGT) activities as described previously (29).

Statistical Analysis.

Variance analysis was performed using ANOVA.

### RESULTS

Introduction of the Pdx1 Gene into FH-hTERT Cells

To induce differentiation of FH-h-TERT cells (20) into insulin-producing cells, a suclone, designated FH-B, was infected with a lentivirus vector containing the Pdx1 and neomycin resistance genes, both expressed from a common promoter using an internal ribosomal entry site (IRES) (FIG. 1a). Cells surviving 16 days of selection in G418 were termed FH-B-TPN (for Telomerase, Pdx1, and Neo). FH-B-TPN cells did not manifest an obvious change in cell morphology. They continued to grow as monolayers on cell culture plastic, which was similar to the parental FH-B cells (FIG. 1b). In confluent cultures, ball-shaped cell clusters could be observed (FIG. 1c). However, the rate of cell proliferation in FH-B-TPN cells declined.

[³H]thymidine incorporation in DNA was reduced 3-fold in FH-B-TPN cells, compared with FH-B cells (2367±394 cpm versus 7381±668 cpm, mean±SEM, n=12, P<0.001).

Changes in Gene Expression Following Pdx1 Expression in FH-B-TPN Cells

The pattern of gene expression in FH-B-TPN cells was analyzed with a representative panel of liver genes expressed in parental FH cells and FH-B cells. These studies included reverse transcription polymerase chain reaction (RT-PCR) as well as histochemical stainings (FIG. 2).

Despite transduction with Pdx1 lentivirus and G418 selection, FH-B-TPN cells continued to express multiple liver genes, including glycogen (hepatocyte marker), and dipeptidyl peptidase IV (DPPIV) and γ-glutamyl transpeptidase (GGT) (biliary markers), which was similar to the parental FH and FH-B cells. FH-B-TPN cells showed differences in transcription factor expression compared with FH and FH-B cells. Some transcription factors expressed in FH cells were extinguished in FH-B and FH-B-TPN cells, e.g., hepatocyte nuclear factor (HNF)-4, GATA-4, and CCAAT-enhancer binding protein (C/EBP)α, whereas C/EBPγ continued to be expressed. The expression of hepatocyte growth factor (HGF) was unchanged in FH-B-TPN cells, while expression of HNF-1β, transforming growth factor (TGF)α, TGFβ1, and TGFβ1R mRNAs, and to a lesser extent GATA-1 mRNA, was downregulated in FH-B-TPN, compared with FH-B cells. Moreover, FH-B-TPN cells expressed GATA-6, which was similar to FH cells. This multilineage gene expression pattern, with expression of hepatocyte markers, biliary markers and GATA-1, which is characteristic of hematopoietic cells, indicated that FH-B-TPN cells retained a stem/progenitor cell phenotype.

Analysis of Pdx1 expression showed that FH-B-TPN cells expressed both rat Pdx1, which was exogenously introduced, and endogenous human Pdx1, which was likely activated by the rat Pdx1 transgene (30,31) (FIG. 3a). Rat Pdx1 shares 88% amino acid homology with the human Pdx1 protein, and was therefore expected to be active in human cells.

Expression of β-cell and pancreatic genes was evaluated in FH-B-TPN cells by RT-PCR analysis (FIG. 3b). Expression of genes encoding two transcription factors found in mature β. cells, BETA2 and NKX6.1, as well as neurogenin 3 (NGN3), a transcription factor found in fetal islet cells, was observed. In contrast, genes for 3 other β-cell transcription factors, NKX2.2, ISL1, and PAX6, were not expressed in FH-B-TPN cells. PAX4, a transcription factor found in embryonic β. cells, was also not expressed in FH-B-TPN cells, although it was present in adult human pancreatic islets.

Of note, human insulin mRNA was expressed in FH-B-TPN cells, along with transcripts for 2 proinsulin processing enzymes, prohormone convertase (PC) 1/3 and PC2, indicating that the Pdx1-modified cells acquired the ability to synthesize and process proinsulin to mature insulin. Transcripts encoding the β-cell protein islet amyloid polypeptide (IAPP) were also detected. Moreover, expression of a major component of dense-core secretory granules, chromogranin A (CGA), was observed, suggesting induction of a regulated secretory pathway in the FH-B-TPN cells, which is not normally present in hepatocytes. Transcripts for another component of the secretory vesicle, synaptogyrin 3 (SYNG3), were present in both FH-B and FH-B-TPN cells. Of the 2 components of the K+ATP channel, SUR1 and KIR6.2, only the latter was expressed in FH-B-TPN cells. In contrast, expression of GLUT2 and GK, which participate in signal-secretion coupling in β-cells, and are also present in mature hepatocytes, could not be detected in FH-B-TPN cells. Glucokinase (GK) transcription was detectable however, when different primers and a different RT-PCR kit was used (see section on promotion of differentiation of FH-B-TPN cells towards the β-cell phenotype., FIG. 10).

Additional transcripts encoding proteins found in non-β. islet cells, as well as in exocrine pancreas, were detected in FH-B-TPN cells. These included glucagon, pancreatic polypeptide (PP), and elastase. Transcripts for somatostatin were not detected. Notably, FH-B-TPN cells expressed glucagon mRNA in the absence of detectable PAX6, which is needed in pancreatic islets for β-cell development and gene expression (32).

Insulin Production Storage, and Regulated Secretion in FH-B-TPN Cells

To determine the proportion of insulin-positive cells within the total FH-B-TPN cell population, PDX1 and insulin expression was analyzed by immunostaining. These studies showed expression of both PDX1 and insulin proteins in the vast majority of cells (FIG. 1d and e). A weak immunostaining for PP was detected in most FH-B-TPN cells, however immunostaining failed to demonstrate glucagon protein in the cells (data not shown), indicating that if glucagon mRNA was correctly translated, the amount of glucagon produced was minuscule. The insulin content of FH-B-TPN cells was found to be 185±38 ng per 1×10⁶ cells (or 100 μg protein). The cells were monitored for insulin content during 38 population doublings following G418 selection without a notable change. Presence of immunostainable insulin demonstrated that FH-B-TPN cells were capable of storing insulin. This ability to store insulin was further established by radioimmunoassay (RIA). The insulin content of FH-B-TPN cells was found to be 150 ng insulin per 1×10⁶ cells.

Insulin secretion in response to glucose was determined by static incubations. Most insulin was released from FH-B-TPN cells in response to stimulation with glucose concentrations between 8 and 20 mM (FIG. 4). This phenotype of regulated insulin secretion following glucose stimulation was maintained in multiple experiments using FH-B-TPN cells after 4-30 population doublings following G418 selection.

FH-B-TPN Cells Normalize Blood Glucose Levels in Hyperglycemic Mice

To determine the ability of FH-B-TPN cells to replace β-cell function, they were transplanted into nonobese diabetic severe combined immunodeficient (NOD-scid) mice, which were treated with streptozotocin (STZ) to eliminate their □cells. Starting from 2 weeks following transplantation, blood glucose levels in the transplanted mice decreased and were stabilized around 160 mg/dl for the remainder of the experiment, during about 8 weeks (FIG. 5), whereas untransplanted mice remained hyperglycemic. Serum insulin levels in the transplanted mice averaged 0.98±0.16 ng per ml, which is within the normal range for mice.

Analysis of human C-peptide in the serum of the transplanted mice detected amounts comparable to those of insulin (1.18±0.26 ng per ml, compared to undetectable levels in the untransplanted mice). These findings demonstrate that the glycemia was normalized by insulin secretion from the transplanted human cells, rather than by islet regeneration. An i.p. glucose tolerance test performed in the transplanted mice showed a clearance rate similar to that of normal NOD-scid mice (FIG. 5b), suggesting that insulin secretion from the cells was regulated by glucose as shown in the cultured cells. Analysis of the mice sacrificed after completion of the experiment, close to 3 months after cell transplantation, did not demonstrate tumors in the peritoneal cavity. These results establish the capacity of FH-B-TPN cells to function as surrogate β cells in vivo.

Promoting Differentiation of FH-B-TPN Cells Towards the β-Cell Phenotype.

To promote further differentiation of FH-B-TPN cells towards a β cell phenotype, the cells were incubated in serum-free medium supplemented with ITS. Following a 6-day incubation, insulin content increased 15-fold, to 2766±232 ng per 1×10⁶ cells. Quantitation of cellular insulin content using an ELISA kit which detects only mature insulin revealed a content of 2580±77 ng per 1×10⁶ cells, indicating that most of the insulin was stored in the cells in a processed form. No insulin was detected in control FH-B cells incubated in the same conditions. An assay of glucose-induced insulin secretion during a 30-minute static incubation demonstrated the same dose response observed in cells grown in regular medium (FIG. 6a).

RT-PCR analysis revealed a large increase in insulin and PC2 mRNA levels, as well as induction of expression of NKX2.2 (FIG. 6b). However, transcripts for GLUT2, GK, and SUR1 were still absent following this treatment (data not shown).

Transplantation of cells following a 6-day incubation in serum-free medium under the renal capsule of STZ-diabetic NOD-scid mice resulted in a rapid restoration of euglycemia (FIG. 6c). Fed serum insulin and human C-peptide levels 7 days following transplantation were 2.18±0.32 and 3.48±0.37 ng per ml, respectively. Removal of the transplanted cells by nephrectomy caused a rapid increase in blood glucose levels, demonstrating that the correction of blood glucose was due to the transplanted cells. These results establish the capacity of FH-B-TPN cells to function as surrogate β cells in vivo.

The effect of a 6-day treatment with 4 nM activin A on cell differentiation was also evaluated. PAX4 and GLUT2 mRNAs appeared following 3 days of treatment, while glucagon mRNA disappeared after an overnight treatment (FIG. 7).

Cells incubated for 6 days in serum-free medium supplemented with both ITS and activin A showed a further doubling of the insulin content, compared with cells incubated in serum-free medium in the absence of ITS alone. In addition, a significant increase was observed under these conditions in the percentage of cells stained for the transcription factors ISL1, NKX6.1, and NKX2.2, while a significant decrease was noted in the percent of cells stained for a marker of precursor islet cells, NGN3 (FIG. 8). Similarly, an increase was observed in the percent of cells stained for GK, PC1/3, and PC2, while PP staining disappeared. No staining for glucagon and somatostatin was detected in cells incubated in either the presence of serum or in serum-free medium supplemented with ITS and activin A (data not shown). These results indicate that a 6-day treatment of FH-B-TPN cells in serum-free medium in the presence of both ITS and activin A promotes differentiation to some extent towards the β-cell phenotype, while suppressing the expression of some non-β-cell islet genes.

Due to the fact that the treated FH-B-TPN cells still deviated from that of normal human beta cells, a further analysis was performed to ascertain whether addition of other soluble factors would further aid in the promotion of the beta cell phenotype.

As seen in Table 3 hereinbelow, out of the 4 factors analyzed for their effect in CM: Act-A, BTC, NA, and exendin-4, only Act-A increased cellular insulin content significantly, approximately 4-fold, compared with cells cultured in regular medium, as judged by RIA. Exendin-4, and to a lesser extent NA, induced a decrease in insulin content of FH-B-TPN cells. A combined treatment with Act-A+BTC+NA did not result in an additive effect, compared with Act-A alone. Similarly, a combination of Act-A with hepatocyte growth factor (HGF) is contraindicated, despite previous reports showing that HGF contributes to the differentiation of insulin-producing cells at a higher concentration (33). The optimal effect of Act-A was achieved at a concentration of 3 nM (FIG. 9).

TABLE 3

| | Insulin content | |
|---|---|---|
| Treatment | (ng/10⁶ cells) | Fold increase |
| CM | 188 ± 19 | 1 |
| Act-A 4 nM in CM (6 d) | 729 ± 55 | 3.9 |
| BTC 4 nM in CM (6 d) | 264 ± 7 | 1.4 |
| NA 5 mM in CM (6 d) | 122 ± 20 | 0.6 |
| Exendin-4 2-8 nM in CM (6 d) | 0 | 0 |
| Act-A 4 nM + HGF 100 pM in CM (6 d) | 254 ± 15 | 1.3 |
| Act-A 4 nM + BTC + NA in CM (6 d) | 578 ± 35 | 3.1 |

To evaluate the effect of Act-A in combination with the SFM treatment, the cells were incubated using protocols and various combinations of the two conditions as detailed in Table 4. The effect of a 3-day treatment with 3 nM Act-A was more pronounced in SFM, compared to CM. When the 3-day Act-A treatment in SFM was preceded by a 6-day incubation in SFM in the absence of Act-A, insulin content was greatly increased, to 33 times that of cells grown in CM. This represented a 2.6-fold increase over the insulin content of cells incubated for the same combined period of 9 days in SFM in the absence of Act-A. This insulin content represents 6% of the cellular protein content, and about 60% of the insulin content of normal human pancreatic islets. When the order was reversed, with the Act-A treatment preceding the incubation in SFM, the resulting insulin content was not much higher than with Act-A alone. The RIA results were confirmed by ELISA analysis, which detects only mature insulin, showing that 95% of stored insulin was in the form of mature protein. Human C-peptide levels in these cells, as analyzed by ELISA, were 5153±180 ng/10⁶ cells. The levels of human C-peptide secreted into the culture medium were 43.5±1.3 ng/10⁶ cells. No insulin was detected in FH-B cells, which did not express Pdx1, when treated under these conditions, suggesting that insulin expression could not be induced by these treatments in the absence of Pdx1.

TABLE 4

| | Insulin content | |
|---|---|---|
| Treatment* | (ng/10⁶ cells) | Fold increase |
| CM | 188 ± 19 | 1 |
| Act-A 3 nM in CM (3 d) | 1399 ± 269 | 7.4 |
| Act-A 3 nM in SFM (3 d) | 2143 ± 179 | 11.4 |
| SFM (9 d) | 2351 ± 281 | 12.5 |
| SFM (6 d) + Act-A 3 nM in SFM (3 d) | 6157 ± 231 | 32.7 |
| Act-A 3 nM in CM (3 d) + SFM (6 d) | 1493 ± 229 | 7.9 |
| Act-A 3 nM in CM (3 d) + CM (6 d) | 1661 ± 319 | 8.8 |

*FH-B-TPN cells were treated with the indicated media for number of days shown in brackets. Insulin content in cell extracts was quantitated by RIA.
Values are mean ± SD (n = 3).

The change in insulin content was accompanied by changes in expression of other genes, as revealed by RT-PCR analyses (FIG. 10). An increase in insulin mRNA levels was induced by all 3 treatments (columns 6-8: 3 nM Act-A for 3 days in CM; SFM for 6 days; SFM for 6 days followed by 3 nM Act-A for 3 days in CM). Among the transcription factor genes analyzed, NeuroD transcripts were induced by all 3 treatments, most notably by SFM followed by Act-A, and Nkx2.2 was highly induced by SFM, and to a lesser extent by SFM followed by Act-A. In contrast, Nkx6.1 transcription was detected in all the conditions studied. Pax6 transcription decreased following incubation in all 3 media, particularly in the two lacking serum. Transcription of the prohormone convertase PC1/3 was significantly elevated only by SFM followed by Act-A, while PC2 transcript levels were not affected.

All 3 treatments resulted in a significant increase in GK transcript levels. Of the 2 non-β islet cell genes analyzed, glucagon expression was decreased in the presence of Act-A, and elevated by SFM lacking Act-A, while PP transcripts, as well as those of the hepatic gene alpha 1 antitrypsin (α1AT), increased in response to Act-A alone, but decreased in the 2 conditions lacking serum. Most of the genes analyzed were not expressed in similarly treated FH-B cells, indicating that in the absence of Pdx1 the culture medium conditions alone were not sufficient for inducing their expression. Notable exceptions were the activation of NeuroD expression following treatment with Act-A, and PC1/3 induction by the SFM treatment followed by Act-A (FIG. 10). These findings were reproducible in multiple independent experiments.

Immunofluorescence analyses comparing cells in CM with cells treated in SFM followed by Act-A showed an increase in the staining intensity for insulin and C-peptide (FIG. 11). In addition, a significant increase in the number of cells stained for NeuroD and NKX2.2 was observed, confirming the RNA analyses. Conversely, this culture condition resulted in the disappearance of PAX6 and PP immunostaining in all analyzed cells. PC1/3 and GK immunostaining was present in all analyzed cells grown in CM, however the staining intensity for PC1/3 increased following the SFM+Act-A treatment. No glucagon or somatostatin staining was visible in cells in either condition.

The cell doubling time, as determined by cell counting, was not affected by Act-A treatment in CM. Incubation in SFM increased doubling time two-fold, resulting in a slower proliferation rate, compared with cells growing in CM, while the SFM+Act-A treatment increased it four-fold. No apoptotic cells were detected using a TUNEL assay under any of the conditions (data not shown).

Insulin secretion was shown to be glucose-responsive in the physiological concentration range in FH-B-TPN cells in both CM and SFM in the presence of Act-A, both during 3 days in CM and following Act-A treatment during the last 3 of 9 days in SFM (FIG. 12). The maximal secretion at 20 mM glucose of cells treated with SFM+Act-A represents 1.1% of their insulin content, which is similar to that of normal islets.

To evaluate the dependence of the differentiated cell phenotype following SFM+Act-A treatment on continuous culture under these conditions, cells were shifted following the treatment to a 10-day period in CM. As seen in FIG. 10, no significant changes were observed between columns 8 and 9 in transcripts of Nkx2.2, GK, PC1/3 and PC2, and no reappearance of glucagon transcripts was observed. In contrast, there was a reduction in the levels of insulin, NeuroD, and Nkx6.1 transcripts, and reappearance of Pax6, PP, and α1AT transcripts. Insulin content was reduced by 41%, to 3626±207 ng/10⁶ cells.

To assess the functional stability of the FH-B-TPN cells in vivo following treatment in vitro with SFM+Act-A, cells were transplanted under the renal capsule of STZ-diabetic NOD-SCID mice. As seen in FIG. 13A, blood glucose levels were lowered from 2 days post-transplantation. Glycemia was normalized thereafter, and stable blood glucose levels were maintained for over 2 months, until the experiment was terminated for histological analyses. No hypoglycemia developed by the end of the experiment. Prior to sacrificing the mice, they were subjected to a glucose tolerance test, which demonstrated a normal rate of glucose clearance (FIG. 13B). Human C-peptide ELISA detected serum levels ranging between 0.31-0.84 ng/ml (compared with 0.27 ng/ml in a human serum control, and no detectable signal in normal mouse serum). Histological analyses detected insulin and human C-peptide immunofluorescence staining in cells positively identified as human using a human-specific anti-heat shock protein (HSP) 27 antibodies with no cross reactivity to mouse (FIG. 13C). No BrdU-labeled cells were detected in the transplants, indicating that little or no cell replication occurred in the transplanted cells at this time point (FIG. 13C).

### CONCLUSION

These results demonstrate that Pdx1 activates the expression of insulin, as well as other β-cell genes, in human fetal liver progenitor cells. In expressing Pdx1 in FH-B-TPN cells, the goal was not only to activate insulin expression, but rather to induce a cellular phenotype along the mature pancreatic β-cell lineage. This would be manifested by insulin production in normal amounts, insulin processing and storage, and appropriate insulin release in response to physiological signals. The results described hereinabove suggest that FH-B-TPN cells have undergone profound changes as a result of Pdx1 expression. The cells of the present invention acquired the ability to produce proinsulin, as shown by insulin mRNA and protein analyses.

The cellular insulin content was increased by up to 33-fold, to over 6% of cellular protein content in FH-B-TP cells incubated in SFM in the presence of Act-a. This represents about 60% of the content of normal human pancreatic islets. These amounts of insulin result from biosynthesis in the cells, rather than uptake from the medium, as judged by the following criteria: 1) no insulin was detected in FH-B cells cultured in the same conditions; 2) insulin was also detected in FH-B-TPN cells cultured in CM which is not supplemented with insulin; 3) insulin mRNA was detected in the cells; 4) human C-peptide was detected in the cells by ELISA and immunofluorescence, in the culture medium, and in the serum of mice transplanted with these cells. The modified cells maintained a normal glucose-induced insulin secretory profile in the physiological concentration range. Induction of insulin expression in these cells was likely due to the rat Pdx1 transgene, as well as to activation of the endogenous human Pdx1 gene by rat Pdx1, as indicated by RT-PCR analysis.

The expression of PC1/3 and PC2 in FH-B-TPN cells suggest that the cells of the present invention possess the ability to process proinsulin to mature insulin. Analysis of insulin content in FH-B-TPN cells by immunofluorescence and RIA demonstrated an ability of these cells to store significant amounts of insulin. While mature liver cells lack a regulated secretory pathway, expression of mRNAs for proteins found in secretory vesicles, such as CGA and SYNG3, suggests that insulin may be stored in vesicles similar to those present in pancreatic β cells. Release of the stored insulin in response to glucose in the physiological concentration range suggested induction of a signal-secretion coupling apparatus in FH-B-TPN cells.

Pdx1 expression did not extinguish liver gene expression in FH-B-TPN cells, as shown by the presence of glycogen, DPPIV and GGT, as well as expression of several liver transcription factor and growth factor genes. However, some differences in expression of these genes following Pdx1 expression were obvious in FH-B-TPN cells, including extinction of HNF-1 β. and reactivation of the GATA-6 transcription factor. In addition, FH-B-TPN cells lost expression of multiple growth factors, including TGFα and TGF β, as well as TGF β receptor.

FH-B-TP cells incubated in SFM in the presence of Act-a, were further differentiated towards the beta cell phenotype as demonstrated by the expression of the beta-cell transcription factors NeuroD and Nkx2.2, and the down-regulated expression of the alpha-cell transcription factor Pax6. Changes in expression of other genes may be as a result of this shift in transcription factor profile, or may be directly effected by the inductive conditions. The resulting up-regulation of glucokinase and PC1/3 expression, and the down-regulation of PP as well as the hepatic marker α1AT, brought the phenotype of FH-B-TPN cells closer to that of normal beta cells. The immunofluorescence analyses demonstrated that the phenotype of the FH-B-TPN cell population is uniform. Presence of transcripts of Ngn3, a transcription factor which in mice is found in embryonic but not in mature pancreatic islets (34), indicated that phenotypically FH-B-TPN cells resembled immature islet precursor cells more closely than mature β cells.

The clearest demonstration of the differentiation of FH-B-TPN cells along the β-cell lineage is their ability to replace β-cell function for long periods of time in vivo. The lag period of over 2 weeks observed in the transplanted mice between the time of transplantation and normalization of blood glucose levels may reflect a need for further cell differentiation in vivo, or for vascularization of the transplanted cells.

The reconstitution of telomerase in FH-B cells (20), which is required for maintaining chromosomal stability during prolonged cell proliferation, permitted the expansion of these cells in culture both before and following Pdx1-induced differentiation. It has been established that FH-B cells maintain normal telomere length for over 300 cell doublings, with no tumorigenic potential in NODscid mice (20).

Indeed, no evidence for neoplasia was revealed in mice transplanted with FH-B-TPN cells 3 months post-transplantation. In conclusion, these studies establish the potential of human liver progenitor cells in expressing insulin and releasing it in a regulated fashion.

### REFERENCES

1. Efrat, S., Fusco-DeMane, D., Lemberg, H., Emran, O. A. & Wang, S. Conditional transformation of a pancreatic β-cell line derived from transgenic mice expressing a tetracycline-regulated oncogene. Proc. Natl. Acad. Sci. USA 92, 3576-3580 (1995).
2. Fleischer, N. et al. Functional analysis of a conditionally-transformed pancreatic β-cell line. Diabetes 47, 1419-1425 (1998).
3. Milo-Landesman, D. et al. Correction of hyperglycemia in diabetic mice transplanted with reversibly-immortalized pancreatic βcells controlled by the tet-on regulatory system. Cell Transplant. 10, 645-650 (2001).
4. de la Tour, D. et al. Beta-cell differentiation from a human pancreatic cell line in vitro and in vivo. Mol. Endocrinol. 15, 476-483 (2001).
5. Soria, B. et al. Insulin-secreting cells derived from embryonic stem cells normalize glycemia in streptozotocin-induced diabetic mice. Diabetes 49, 157-162 (2000).
6. Assady, S. et al. Insulin production by human embryonic stem cells. Diabetes 50, 1691-1697 (2001).
7. Lumelsky, N. et al. Differentiation of embryonic stem cells to insulin-secreting structures similar to pancreatic islets. Science 292, 1389-1394 (2001).
8. Clarke, D. L. et al. Generalized potential of adult neural stem cells. Science 288, 1660-1663 (2000).
9. Jiang, Y. et al. Pluripotency of mesenchymal stem cells derived from adult marrow. Nature 418, 41-49 (2002).
10. Jonsson, J., Carlsson, L., Edlund, T. & Edlund, H. Insulin-promoter-factor 1 is required for pancreas development in mice. Nature 371, 606-609 (1994).
11. Ahlgren, U., Jonsson, J., Jonsson, L., Simu, K. & Edlund, H. Beta-cell-specific inactivation of the mouse Ipf1/Pdx1 gene results in loss of the beta-cell phenotype and maturity onset diabetes. Genes Dev. 12, 1763-1768 (1998).
12. Ferber, S. et al. Pancreatic and Duodenal homeobox gene I induces expression of insulin genes in liver and ameliorates streptozotocin-induced hyperglycemia. Nat. Med. 6, 568-572 (2000).
13. Horb, M. E., Shen, C. N., Tosh, D. & Slack, J. M. (2003) Curr. Biol. 13, 105-115.
14. Kojima, H., Nakamura, T., Fujita, Y., Kishi, A., Fujimiya, M., Yamada, S., Kudo, M., Nishio, Y., Maegawa, H. et al. (2002) Diabetes 51, 1398-1408.
14b. Ferber S., et al.,(2005) PNAS, 102, 7964-7969.
15. Ramiya, V. K., Maraist, M., Arfors, K. E., Schatz, D. A., Peck, A. B. & Cornelius, J. G. (2000) Nat. Med. 6, 278-282.
16. Bonner-Weir, S., Taneja, M., Weir, G. C., Tatarkiewic, K., Song, K. H., Sharma, A. & O'Neil, J. J. (2000) Proc. Natl. Acad. Sci. USA 97, 7999-8004.
17. Suzuki, A., Zheng, Yw., Kaneko, S., Onodera, M., Fukao, K., Nakauchi, H. & Taniguchi, H. (2002) J. Cell Biol. 156, 173-184.
18. Yang, L., Li, S., Hatch, H., Ahrens, K., Cornelius, J. G., Petersen, B. E. & Peck, A. B. (2002) Proc. Natl. Acad. Sci. USA 99, 8078-8083.
19. Malhi, H., Irani, A. N., Gagandeep, S. & Gupta, S. (2002) J. Cell. Sci. 115, 2679-2688.
20. Wege, H., Le, H. T., Chui, M. S., Liu, L., Wu, J., Giri, R. K., Malhi, H., Sappal, B. S., Kumaran, V., Gupta, S. & Zern, M. A. (2003) Gastroenterology 124, 432-444.
21. Olsen, J. C. (1998) Gene Ther. 5, 1481-1487.
22. Rohll, J. B., Mitrophanous, K. A., Martin-Rendon, E., Ellard, F. M., Radcliffe, P. A., Mazarakis, N. D. & Kingsman, S. M. (2002) Methods Enzymol. 346, 466-500.
23. Miller, C. P., McGehee, R. E. Jr. & Habener, J. F. (1994) EMBO J 13, 1145-1156.
24. Adra, C. N., Boer, P. H. & McBurney, M. W. (1987) Gene 60, 65-74.
25. Jang, S. K., Krausslich, H. G., Nicklin, M. J., Duke, G. M., Palmenberg, A. C. & Wimmer, E. (1988) J. Virol. 62, 2636-2643.
26. Mitrophanous, K., Yoon, S., Rohll, J., Patil, D., Wilkes, F., Kim, V., Kingsman, S., Kingsman, A. & Mazarakis, N. (1999) Gene Ther. 6, 1808-1818.
27. Zufferey, R., Donello, J. E, Trono, D. & Hope, T. J. (1999) J. Virol. 73, 2886-2892.
28. Naldini, L., Blomer, U., Gage, F. H., Trono. D. & Verma, I. M. (1996) Proc. Natl. Acad. Sci. USA 93, 11382-11388.
28b. Berkovich I, Efrat S (2001) Diabetes 50: 2260-2267.
29. Ott, M., Rajvanshi, P., Sokhi, R., Alpini, G., Aragona, E., Dabeva, M, Shafritz, D. A. & Gupta, S. (1999) J. Pathol. 187, 365-373.
30. McKinnon, C. M. & Docherty, K. (2001) Diabetologia 44, 1203-1214.
31. Marshak, S., Ben-Shushan, E., Shoshkes, M., Havin, L., Cerasi, E. & Melloul, D. (2001) Diabetes 50 (Suppl. 1), S37-S38.
32. Dohrmann, C., Gruss, P. & Lemaire, L. (2000) Mech. Dev. 92, 47-54.
33. Zhang Y Q, Kanzaki M, Furukawa M, Shibata H, Ozeki M, Kojima I (1999) Diabetologia 42:719-727.
34. Gradwohl, G., Dierich, A., LeMeur, M. & Guillemot, F. (2000) Proc. Natl. Acad. Sci. USA 97, 1607-1611.

All publications mentioned in the above specification, and references cited in said publications, are herein incorporated by reference. Various modifications and variations of the described methods and system of the present invention will be apparent to those skilled in the art without departing from the scope and spirit of the present invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A human hepatic cell capable of endogenous insulin production wherein said insulin production is at least 50% of that of a normal β cell.

2. The human hepatic cell of claim 1, wherein said insulin production is up-regulated in the presence of activin A and a serum free medium.

3. The human hepatic cell of claim 1, being genetically modified.

4. The human hepatic cell of claim 3, wherein said genetically modified cell expresses at least one pancreatic beta cell gene.

5. The human hepatic cell of claim 4, wherein said at least one pancreatic beta cell gene is a transcription factor.

6. The human hepatic cell of claim 5, wherein said transcription factor is selected from the group consisting of PD-X, NKX6.1, neurogenin 3, beta 2 and E2A.

7. The human hepatic cell of claim 6, wherein said transcription factor is PD-X.

8. The human hepatic cell of claim 1, wherein said endogenous insulin is secreted.

9. The human hepatic cell of claim 1, wherein said endogenous insulin production is glucose regulated.

10. The human hepatic cell of claim 1, wherein the human hepatic cell is derived from a fetal progenitor liver cell.

11. The human hepatic cell of claim 10, wherein said fetal progenitor liver cell is derived from an epithelial fetal progenitor liver cell.

12. The human hepatic cell of claim 1, wherein the human hepatic cell is derived from an adult hepatic stem cell.

13. The human hepatic cell of claim 1, wherein the human hepatic cell is a differentiated adult hepatic cell.

14. The human hepatic cell of claim 1, wherein the human hepatic cell is immortalized.

15. A method of up-regulating endogenous insulin production in a hepatic cell, the method comprising:
(a) genetically modifying the human hepatic cell to express at least one beta cell gene; and
(b) culturing said genetically modified hepatic cell in the presence of serum-free medium and activin A to thereby up-regulate endogenous insulin production in the human hepatic cell.

16. The method of claim 15, further comprising isolating the hepatic cell expressing said at least one beta cell gene, above a predetermined threshold.

17. The method of claim 15, wherein said activin A is provided at a concentration range of 1-8 nM.

18. The method of claim 17, wherein said activin A is provided at a concentration of 3 nM.

19. The method of claim 15, further comprising immortalising the hepatic cell prior to, concomitant with or following step (a).

20. The method of claim 19, wherein said immortalizing is effected by introducing a telomerase gene into the hepatic cell.

21. Use of the human hepatic cell of claim 1 as a medicament.

22. A method of treating type I diabetes in a subject in need thereof, the method comprising administering to the subject the human hepatic cell of claim 1, thereby treating type 1 diabetes in the subject in need thereof.

23. A cell culture comprising the human hepatic cells of claim 1, activin A and a serum free medium.


SEQUENCE LISTING (96 sequences)

SEQ ID NO: 1 - DNA, 25 bp, Artificial sequence
  Single strand DNA oligonucleotide
caaggacccatgcgcgttccagcga

SEQ ID NO: 2 - DNA, 27 bp, Artificial sequence
  Single strand DNA oligonucleotide
gaactccttctccagctctagcagctg

SEQ ID NO: 3 - DNA, 25 bp, Artificial sequence
  Single strand DNA oligonucleotide
caaggacccatgcgcgttccagcga

SEQ ID NO: 4 - DNA, 28 bp, Artificial sequence
  Single strand DNA oligonucleotide
gaactcccttctccagctctagcagctg

SEQ ID NO: 5 - DNA, 23 bp, Artificial sequence
  Single strand DNA oligonucleotide
cctgagcagaaccaggacatgcc

SEQ ID NO: 6 - DNA, 25 bp, Artificial sequence
  Single strand DNA oligonucleotide
atcaaaggaagggctggtgcaatca

SEQ ID NO: 7 - DNA, 24 bp, Artificial sequence
  Single strand DNA oligonucleotide
ctcctcctcgtcctcgtcgtcgtc

SEQ ID NO: 8 - DNA, 22 bp, Artificial sequence
  Single strand DNA oligonucleotide
cttgacctgactctctgtcatc

SEQ ID NO: 9 - DNA, 23 bp, Artificial sequence
  Single strand DNA oligonucleotide
cggacaatgacaaggagaccccg

SEQ ID NO: 10 - DNA, 25 bp, Artificial sequence
  Single strand DNA oligonucleotide
cgctcaccaagtccactgctgctgg

SEQ ID NO: 11 - DNA, 24 bp, Artificial sequence
  Single strand DNA oligonucleotide
gtgcggagtgtaatcagtatttgg

SEQ ID NO: 12 - DNA, 24 bp, Artificial sequence
  Single strand DNA oligonucleotide
gtcatctctaccagttgctccttc

SEQ ID NO: 13 - DNA, 25 bp, Artificial sequence
  Single strand DNA oligonucleotide
gctgcatcagaagaggccatcaggc

SEQ ID NO: 14 - DNA, 25 bp, Artificial sequence
  Single strand DNA oligonucleotide
gcgtctagttgcagtagttctccag

SEQ ID NO: 15 - DNA, 27 bp, Artificial sequence
  Single strand DNA oligonucleotide
ttggctgaaagagaacgggatacatct

SEQ ID NO: 16 - DNA, 27 bp, Artificial sequence
  Single strand DNA oligonucleotide
acttctttggtgattgctttggcggtg

SEQ ID NO: 17 - DNA, 24 bp, Artificial sequence
  Single strand DNA oligonucleotide
gcatcaagcacagacctacactcg

SEQ ID NO: 18 - DNA, 25 bp, Artificial sequence
  Single strand DNA oligonucleotide
gagacacaaccacccttcatcgttc

SEQ ID NO: 19 - DNA, 22 bp, Artificial sequence
  Single strand DNA oligonucleotide
ctcactgctcaagaagacatgg

SEQ ID NO: 20 - DNA, 22 bp, Artificial sequence
  Single strand DNA oligonucleotide
ctgggtaacagggatcaaacag

SEQ ID NO: 21 - DNA, 24 bp, Artificial sequence
  Single strand DNA oligonucleotide
gccatccttcagtctctgctactc

SEQ ID NO: 22 - DNA, 26 bp, Artificial sequence
  Single strand DNA oligonucleotide
gctatcatgctcacataactcatcca

SEQ ID NO: 23 - DNA, 24 bp, Artificial sequence
  Single strand DNA oligonucleotide
gacgagttcctgctggagtatgac

SEQ ID NO: 24 - DNA, 27 bp, Artificial sequence
  Single strand DNA oligonucleotide
gactcgatgaaggtgatctcgcagctg

SEQ ID NO: 25 - DNA, 28 bp, Artificial sequence
  Single strand DNA oligonucleotide
gtgcacatccaccacagcacatggcttc

SEQ ID NO: 26 - DNA, 28 bp, Artificial sequence
  Single strand DNA oligonucleotide
gtgtcttgaagaagatgtatctcctcac

SEQ ID NO: 27 - DNA, 21 bp, Artificial sequence
  Single strand DNA oligonucleotide
cgctggtggacctcaagtggc

SEQ ID NO: 28 - DNA, 22 bp, Artificial sequence
  Single strand DNA oligonucleotide
cctcggggctggtggtcttgcg

SEQ ID NO: 29 - DNA, 24 bp, Artificial sequence
  Single strand DNA oligonucleotide
gagagagccactgaattacttgcc

SEQ ID NO: 30 - DNA, 25 bp, Artificial sequence
  Single strand DNA oligonucleotide
cctgaccttatcgtgatctgcctgc

SEQ ID NO: 31 - DNA, 24 bp, Artificial sequence
  Single strand DNA oligonucleotide
ggtagtgactgtctcgtttctgtc

SEQ ID NO: 32 - DNA, 25 bp, Artificial sequence
  Single strand DNA oligonucleotide
agctatgcagagggactccaacctg

SEQ ID NO: 33 - DNA, 22 bp, Artificial sequence
  Single strand DNA oligonucleotide
cggacagttccatgaagctctc

SEQ ID NO: 34 - DNA, 22 bp, Artificial sequence
  Single strand DNA oligonucleotide
gagtcaggagtaggagacaagg

SEQ ID NO: 35 - DNA, 23 bp, Artificial sequence
  Single strand DNA oligonucleotide
actgagcaagcagcgacggagtc

SEQ ID NO: 36 - DNA, 23 bp, Artificial sequence
  Single strand DNA oligonucleotide
gcacccacagccgagcgacagac

SEQ ID NO: 37 - DNA, 27 bp, Artificial sequence
  Single strand DNA oligonucleotide
cacctctctgcctgaggacacggtgag

SEQ ID NO: 38 - DNA, 28 bp, Artificial sequence
  Single strand DNA oligonucleotide
ctgcctcattccaagccatacagtagtg

SEQ ID NO: 39 - DNA, 23 bp, Artificial sequence
  Single strand DNA oligonucleotide
cagtcacagcggagtgaatcagc

SEQ ID NO: 40 - DNA, 23 bp, Artificial sequence
  Single strand DNA oligonucleotide
gccatcttgcgtaggttgccctg

SEQ ID NO: 41 - DNA, 27 bp, Artificial sequence
  Single strand DNA oligonucleotide
gaattcattgcttggctggtgaaaggc

SEQ ID NO: 42 - DNA, 27 bp, Artificial sequence
  Single strand DNA oligonucleotide
catttcaaacatcccacgtggcatgca

SEQ ID NO: 43 - DNA, 24 bp, Artificial sequence
  Single strand DNA oligonucleotide
ctgctgctcctgtccacctgcgtg

SEQ ID NO: 44 - DNA, 24 bp, Artificial sequence
  Single strand DNA oligonucleotide
ctccgagaaggccagcgtgtcctc

SEQ ID NO: 45 - DNA, 27 bp, Artificial sequence
  Single strand DNA oligonucleotide
cgtcagtttctgcagaagtccctggct

SEQ ID NO: 46 - DNA, 27 bp, Artificial sequence
  Single strand DNA oligonucleotide
ccatagccgggtttgagttagcagatc

SEQ ID NO: 47 - DNA, 24 bp, Artificial sequence
  Single strand DNA oligonucleotide
gtgatgacagctgctcactgcgtg

SEQ ID NO: 48 - DNA, 26 bp, Artificial sequence
  Single strand DNA oligonucleotide
catctccaccagcacacaccatggtg

SEQ ID NO: 49 - DNA, 20 bp, Artificial sequence
  Single strand DNA oligonucleotide
cttgagatggaagcaaagag

SEQ ID NO: 50 - DNA, 21 bp, Artificial sequence
  Single strand DNA oligonucleotide
cttcactctgggcaatcctca

SEQ ID NO: 51 - DNA, 22 bp, Artificial sequence
  Single strand DNA oligonucleotide
caagaagtcggtggacaagaac

SEQ ID NO: 52 - DNA, 22 bp, Artificial sequence
  Single strand DNA oligonucleotide
cctcatcttagacgcaccaagt

SEQ ID NO: 53 - DNA, 17 bp, Artificial sequence
  Single strand DNA oligonucleotide
gcaacgccgagagagga

SEQ ID NO: 54 - DNA, 17 bp, Artificial sequence
  Single strand DNA oligonucleotide
tgtcctgcattgtcgcc

SEQ ID NO: 55 - DNA, 23 bp, Artificial sequence
  Single strand DNA oligonucleotide
gaaacaatgagatcacttcctcc

SEQ ID NO: 56 - DNA, 23 bp, Artificial sequence
  Single strand DNA oligonucleotide
ctttgtgcaattgccatgactcc

SEQ ID NO: 57 - DNA, 27 bp, Artificial sequence
  Single strand DNA oligonucleotide
ctgctcggagccacaaagagatccatg

SEQ ID NO: 58 - DNA, 25 bp, Artificial sequence
  Single strand DNA oligonucleotide
atcatctgccacgtgatgctctgca

SEQ ID NO: 59 - DNA, 19 bp, Artificial sequence
  Single strand DNA oligonucleotide
cagtctttcaggtgtaccc

SEQ ID NO: 60 - DNA, 23 bp, Artificial sequence
  Single strand DNA oligonucleotide
gagtgatgatgaaggcagtgcag

SEQ ID NO: 61 - DNA, 20 bp, Artificial sequence
  Single strand DNA oligonucleotide
tccctcttccctcctcaaat

SEQ ID NO: 62 - DNA, 20 bp, Artificial sequence
  Single strand DNA oligonucleotide
ttcccctaaccagattgtcg

SEQ ID NO: 63 - DNA, 20 bp, Artificial sequence
  Single strand DNA oligonucleotide
gagtggaagggaagggcgag

SEQ ID NO: 64 - DNA, 22 bp, Artificial sequence
  Single strand DNA oligonucleotide
gaagaagcacatgatttgggac

SEQ ID NO: 65 - DNA, 18 bp, Artificial sequence
  Single strand DNA oligonucleotide
atggtcccctcggctgga

SEQ ID NO: 66 - DNA, 22 bp, Artificial sequence
  Single strand DNA oligonucleotide
ggcctgcttcttctggctggca

SEQ ID NO: 67 - DNA, 21 bp, Artificial sequence
  Single strand DNA oligonucleotide
aggagccagcctgaatgatga

SEQ ID NO: 68 - DNA, 23 bp, Artificial sequence
  Single strand DNA oligonucleotide
ccctctgatgtcccaagattagc

SEQ ID NO: 69 - DNA, 22 bp, Artificial sequence
  Single strand DNA oligonucleotide
gccctggacaccaactgttgct

SEQ ID NO: 70 - DNA, 22 bp, Artificial sequence
  Single strand DNA oligonucleotide
aggctccaaatgtaggggcagg

SEQ ID NO: 71 - DNA, 20 bp, Artificial sequence
  Single strand DNA oligonucleotide
cgtgctgacatctatgcaat

SEQ ID NO: 72 - DNA, 19 bp, Artificial sequence
  Single strand DNA oligonucleotide
agctgctccattggcatac

SEQ ID NO: 73 - DNA, 18 bp, Artificial sequence
  Single strand DNA oligonucleotide
ccatggagaaggctgggg

SEQ ID NO: 74 - DNA, 20 bp, Artificial sequence
  Single strand DNA oligonucleotide
caaagttgtcatggatgacc

SEQ ID NO: 75 - DNA, 21 bp, Artificial sequence
  Single strand DNA oligonucleotide
acacgagacccactttttccg

SEQ ID NO: 76 - DNA, 23 bp, Artificial sequence
  Single strand DNA oligonucleotide
tgctggacttgtgcttcttcaac

SEQ ID NO: 77 - DNA, 24 bp, Artificial sequence
  Single strand DNA oligonucleotide
aagaactacatctgggctctgtcg

SEQ ID NO: 78 - DNA, 20 bp, Artificial sequence
  Single strand DNA oligonucleotide
gctgaggggtccatcaaagg

SEQ ID NO: 79 - DNA, 25 bp, Artificial sequence
  Single strand DNA oligonucleotide
ggcatcactaaggtcttcagcaatg

SEQ ID NO: 80 - DNA, 23 bp, Artificial sequence
  Single strand DNA oligonucleotide
gagcgagaggcagttatttttgg

SEQ ID NO: 81 - DNA, 20 bp, Artificial sequence
  Single strand DNA oligonucleotide
tctgaaccttgggagagggc

SEQ ID NO: 82 - DNA, 22 bp, Artificial sequence
  Single strand DNA oligonucleotide
ggtcattttggcaacaatcacc

SEQ ID NO: 83 - DNA, 20 bp, Artificial sequence
  Single strand DNA oligonucleotide
aaccaacacctgtgcggctc

SEQ ID NO: 84 - DNA, 23 bp, Artificial sequence
  Single strand DNA oligonucleotide
gggctttattccatctctctcgg

SEQ ID NO: 85 - DNA, 24 bp, Artificial sequence
  Single strand DNA oligonucleotide
ctcctaaaagacttgcggaatcac

SEQ ID NO: 86 - DNA, 25 bp, Artificial sequence
  Single strand DNA oligonucleotide
tccacacaggcactaagaaagactg

SEQ ID NO: 87 - DNA, 22 bp, Artificial sequence
  Single strand DNA oligonucleotide
gcgggattaccagtccaagttg

SEQ ID NO: 88 - DNA, 21 bp, Artificial sequence
  Single strand DNA oligonucleotide
tgtgctttcagagatgtggcg

SEQ ID NO: 89 - DNA, 19 bp, Artificial sequence
  Single strand DNA oligonucleotide
tcactgtgggcgtggatgg

SEQ ID NO: 90 - DNA, 22 bp, Artificial sequence
  Single strand DNA oligonucleotide
accgaaaaactgagggaagagg

SEQ ID NO: 91 - DNA, 25 bp, Artificial sequence
  Single strand DNA oligonucleotide
gccaaatggagaagagaagaaaaac

SEQ ID NO: 92 - DNA, 19 bp, Artificial sequence
  Single strand DNA oligonucleotide
gttgaagtggtgcccgagg

SEQ ID NO: 93 - DNA, 24 bp, Artificial sequence
  Single strand DNA oligonucleotide
cgttcccttcaagacacagaggag

SEQ ID NO: 94 - DNA, 20 bp, Artificial sequence
  Single strand DNA oligonucleotide
tccctggcggcaagattatc

SEQ ID NO: 95 - DNA, 22 bp, Artificial sequence
  Single strand DNA oligonucleotide
caatgccacaccagagcagatg

SEQ ID NO: 96 - DNA, 18 bp, Artificial sequence
  Single strand DNA oligonucleotide
tgggagcagggagcaagc